(19) United States
(12) Patent Application Publication
(10) Pub. No.: US 2026/0028624 A1
(43) Pub. Date: Jan. 29, 2026

(54) AAV-MEDIATED DELIVERY OF OSTEOBLAST/OSTEOCLAST-REGULATING MIRNAS FOR OSTEOPOROSIS THERAPY

(71) Applicant: University of Massachusetts, Westborough, MA (US)

(72) Inventors: Jae-Hyuck Shim, Worcester, MA (US); Guangping Gao, Worcester, MA (US); Aijaz Ahmad John Bhat, Worcester, MA (US); Jun Xie, Worcester, MA (US); Yeon-Suk Yang, Worcester, MA (US)

(73) Assignee: University of Massachusetts, Westborough, MA (US)

(21) Appl. No.: 18/997,260

(22) PCT Filed: Jul. 18, 2023
(86) PCT No.: PCT/US2023/070392

§ 371 Date: Jan. 21, 2025

Related U.S. Application Data
(60) Provisional application No. 63/391,456, filed on Jul. 22, 2022.

(51) Int. Cl. C12N 15/113; A61P 19/08; C12N 15/86

(52) U.S. Cl. CPC C12N 15/113; A61P 19/08; C12N 15/86; C12N 2310/141; C12N 2750/14143

(57) ABSTRACT

Aspects of the disclosure relate to compositions and methods for modulating bone growth, for example by increasing osteogenesis and/or decreasing osteoclastogenesis. The disclosure is based, in part, on recombinant adeno-associated virus (rAAV) vectors encoding microRNAs or miRNA inhibitors that inhibit endogenous miR-214-3p and/or mediate overexpression of miR-34a-5p in osteoblasts and osteoclasts. In some embodiments, compositions described by the disclosure are useful for treating certain bone diseases or disorders, such as osteoporosis.

### RELATED APPLICATIONS

This application is a national stage filing under 35 U.S.C. § 371 of international PCT application PCT/US2023/070392, filed Jul. 18, 2023, which claims priority under 35 U.S.C. § 119 (e) to U.S. provisional patent application, U.S. Ser. No. 63/391,456, filed Jul. 22, 2022, the entire contents of each of which are incorporated by reference herein.

### Reference to an Electronic Sequence Listing

The contents of the electronic sequence listing (U012070177US01-SEQ-KZM.xml; Size: 43,371 bytes; and Date of Creation: Dec. 3, 2024) is herein incorporated by reference in its entirety.

### BACKGROUND

Osteoporosis occurs due to a dysregulation in bone remodeling, a process requiring both bone-forming osteoblasts and bone-resorbing osteoclasts. Current leading osteoporosis therapies suppress osteoclast-mediated bone resorption but show limited therapeutic effects because osteoblast-mediated bone formation decreases concurrently.

### SUMMARY

Aspects of the disclosure relate to compositions and methods for modulating bone growth, for example by increasing osteogenesis and/or decreasing osteoclastogenesis. The disclosure is based, in part, on recombinant adeno-associated viruses (rAAVs) encoding microRNAs (miRNAs or miRs) or miRNA inhibitors that inhibit endogenous miR-214-3p and/or mediate overexpression of miR-34a-5p in osteoblasts and osteoclasts. In some embodiments, the disclosure provides methods for treating certain bone diseases or disorders, such as osteoporosis, using the rAAVs.

Accordingly, in some aspects, the disclosure provides a nucleic acid comprising a transgene encoding one or more miR-34-5p microRNAs (miRNAs) flanked by adeno-associated virus (AAV) inverted terminal repeats (ITRs).

In some embodiments, a transgene encodes 2, 3, 4, or 5 miR-34-5p miRNAs, each miRNA having the sequence set forth in SEQ ID NO: 1. In some embodiments, each of the one or more miRNAs is encoded by the nucleic acid sequence set forth in SEQ ID NO: 2. In some embodiments, the miRNAs are encoded by the sequence set forth in SEQ ID NO: 3.

In some embodiments, a transgene further comprises a promoter. In some embodiments, a promoter is a RNA polymerase II (RNA pol II) or RNA polymerase III (RNA pol III) promoter. In some embodiments, a promoter comprises a chicken beta-actin (CBA) promoter or a U6 promoter.

In some embodiments, AAV ITRs are AAV2 ITRs. In some embodiments, one or more AAV ITRs are truncated ITRs, for example mutant ITRs (mTRs) or delta ITRs.

In some embodiments, a nucleic acid comprises the sequence set forth in SEQ ID NO: 4.

In some aspects, the disclosure provides a recombinant adeno-associated virus (rAAV) comprising a nucleic acid comprising a transgene encoding one or more miR-34-5p microRNAs (miRNAs) flanked by adeno-associated virus (AAV) inverted terminal repeats (ITRs); and an AAV capsid protein.

In some embodiments, an AAV capsid protein is an AAV9 capsid protein. In some embodiments, an AAV capsid protein is a bone-targeting AAV capsid protein.

In some aspects, the disclosure provides a nucleic acid comprising a transgene encoding a miRNA inhibitor that inhibits expression of miR-214-3p in a subject flanked by adeno-associated virus (AAV) inverted terminal repeats (ITRs).

In some embodiments, a miRNA inhibitor is a tough decoy (TuD) miRNA inhibitor. In some embodiments, a TuD miRNA inhibitor comprises 2 or 3 miR-214-3p binding sites. In some embodiments, each of the miR-214-3p binding sites comprises the sequence set forth in SEQ ID NO: 5.

In some embodiments, a transgene further comprises a promoter. In some embodiments, a promoter is a RNA polymerase II (RNA pol II) or RNA polymerase III (RNA pol III) promoter. In some embodiments, a promoter comprises a chicken beta-actin (CBA) promoter or a U6 promoter.

In some embodiments, AAV ITRs are AAV2 ITRs. In some embodiments, one or more AAV ITRs are truncated ITRs, for example mutant ITRs (mTRs) or delta ITRs.

In some embodiments, a nucleic acid comprises the sequence set forth in SEQ ID NO: 6.

In some aspects, the disclosure provides a recombinant adeno-associated virus (rAAV) comprising a nucleic acid comprising a transgene encoding a miRNA inhibitor that inhibits expression of miR-214-3p in a subject flanked by adeno-associated virus (AAV) inverted terminal repeats (ITRs); and an AAV capsid protein.

In some embodiments, an AAV capsid protein is an AAV9 capsid protein. In some embodiments, an AAV capsid protein is a bone-targeting AAV capsid protein.

In some aspects, the disclosure provides a method of inhibiting bone loss in a subject, the method comprising administering to a subject having bone loss a nucleic acid or rAAV as described by the disclosure.

In some aspects, the disclosure provides a method of increasing bone growth in a subject, the method comprising administering to a subject having increased bone density a nucleic acid or rAAV as described by the disclosure.

In some aspects, the disclosure provides a method for treating or preventing osteoporosis in a subject, the method comprising administering to a subject having, suspected of having, or at risk of having osteoporosis an isolated nucleic acid or rAAV as described by the disclosure.

In some embodiments, administration comprises systemic administration or local administration. In some embodiments, administration comprises injection. In some embodiments, the injection is intravenous injection.

In some embodiments, a subject is a mammal. In some embodiments, a subject is a human.

### BRIEF DESCRIPTION OF DRAWINGS

FIGS. 1A-1K show representative data for rAAV9 carrying miR-214-3p or miR-214-3p TuD on osteoblast or osteoclast differentiation. (A) Diagram showing AAV vector genome that contains miR-214-3p and EGFP gene (top) or miR-214-3p TuD and Guassia luciferase gene (gLuc, bottom). CBA, CMV enhancer/chicken β-actin promoter. (B) Control (ctrl) or miR-214-3p plasmid was transfected into HEK293 cells, and 48 hours later, expression of miR-214-3p was measured by RT-PCR and normalized to U6. (C) 748 HEK293 cells were transfected with ctrl-sensor or miR-214-3p-sensor plasmid in the absence or presence of miR-214-3p plasmid, and 48 hours later, β-galactosidase activity was measured and normalized to Firefly luciferase. (D) The miR-214-3p plasmid was transfected into HEK293 cells along with the miR-214-3p-sensor plasmid and increasing concentrations of the miR-214-3p TuD plasmid. After 48 hours, β-galactosidase activity was measured and normalized to firefly luciferase. (E-G) Mouse bone marrow-derived stromal cells (BMSCs) were transduced with rAAV9 carrying ctrl, miR-214-3p, or miR-214-3p TuD for two days and cultured under osteogenic conditions. ALP staining and activity (E) and expression of Bglap and Ibsp (F) were assessed at day 6 of culture. (G) Computational analysis showing the complementarities of miR-214-3p to the 3′-UTR of Atf4 (left) (SEQ ID NO: 26, 11). mRNA levels of Atf4 in miR-214-3p-expressing BMSCs were assessed by RT-PCR (right). (H-K) Two days after treatment with M-CSF and RANKL, mouse bone marrow monocytes (BMMs) were transduced with Raav9 carrying ctrl, miR-214-3p, or miR-214-3p TuD, cultured under osteoclast differentiation conditions, and stained for TRAP. Representative images of TRAP-stained osteoclasts are shown (H), and the number of TRAP-positive osteoclasts assessed (I). Mrna levels of Rank and Acp5 were measured by RT763 PCR and normalized to Actb (J). (K) The predicted consequential pairing of Pten 3′-UTR with miR-214-3p is shown (top) (SEQ ID NOs: 27,11) and Pten expression as measured by RT-PCR (bottom). Scale bar: H, 400 μm. Values represent mean±SD: ns, not significant; * P<0.05; ** P<0.01; *** P<0.001; and **** P<0.0001 by an unpaired two-tailed Student's t-test (B, G, K) and one-way ANOVA test (C-F, I, J).

FIGS. 2A-2K show representative data for rAAV9 carrying miR-769 34a-5p or miR-34a-5p TuD on osteoblast or osteoclast differentiation. (A) Diagram showing AAV vector genome that contains miR-34a-5p and EGFP gene (top) or miR-34a-5p TuD and gLuc reporter gene (bottom). (B) A control (ctrl) or miR-34a-5p plasmid was transfected into HEK293 cells, and 48 hours later, expression of miR-34a-5p was measured by RT-PCR and normalized to U6. (C) HEK293 cells were transfected with ctrl-sensor or miR-34a-5p-sensor plasmid in the absence or presence of miR-34a-5p plasmid, and 48 hours later, β-galactosidase activity was measured and normalized to firefly luciferase. (D) The miR-34a-5p plasmid was transfected into HEK293 cells along with the miR-34a-5p-sensor plasmid with increasing concentrations of miR-34a-5p TuD plasmid. After 48 hours,-galactosidase activity was measured and normalized to firefly luciferase. (E-G) Mouse BMSCs were transduced with rAAV9 carrying ctrl, miR-34a-5p, or miR-34a-5p TuD for two days and cultured under osteogenic conditions. ALP staining and activity (E) and expression of Runx2 and Sp7 (F) were assessed at day 6 of culture. (G) Computational analysis showing the complementarities of miR-34a-5p to the 3′-UTR of Notch 1 (left) (SEQ ID NOs: 28, 9). mRNA levels of Notch 1 in miR-34a-5p-expressing BMSCs were assessed by RT-PCR (right). (H-K) Two days after treatment with M-CSF and RANKL, BMMs were transduced with rAAV9 carrying ctrl, miR-34a-5p, or miR-34a-5p TuD, cultured under osteoclast differentiation conditions, and stained for TRAP. Representative images of TRAP-stained osteoclasts (H) and numbers of TRAP-positive osteoclasts were quantitated (I). mRNA levels of Ctsk and Trap were measured by RT-PCR and normalized to Actb (J). (K) The predicted consequential pairing of Tgif2 3′-UTR with miR-34a-5p is shown (top) (SEQ ID NOs: 29, 9) and Tgif2 expression as measured by RT-PCR (bottom). Scale bar: H, 400 μm. Values represent mean+SD: ns, not significant; * P<0.05; ** P<0.01; *** P<0.001; and **** P<0.0001 by an unpaired two-tailed Student's t-test (B, G, K) and one-way ANOVA test (C-F, I, J).

FIGS. 3A-3G show representative data for systemic delivery of rAAV9 carrying miR-214-3p or miR-34a-5p TuD results in low bone mass in mice. (A) Diagram of the study and treatment methods. 10-week-old healthy mice were i.v. injected with rAAV9 (5×10¹³ kg/vg) carrying ctrl, miR-214-3p, or miR-34a-5p TuD and eight weeks later, skeletal analysis was performed. (B, E) Expression of miR-214-3p (B) or miR-34a-5p (E) in the tibia and serum was measured by RT-PCR and normalized to U6 (n=5). (C, D, F, G) Femoral bone mass was assessed by microCT. Representative 3D reconstruction (C, F) and relative quantification (D, G) are displayed (n=5). Tra. BV.TV: trabecular bone volume/total volume, Tra. Th: trabecular thickness, Tra. N: trabecular number per cubic millimeter, Tra. Sp: trabecular space. Scale bars: C, F, 400 μm. Values represent mean±SD: * P<0.05; ** P<0.01; and *** P<0.001 by an unpaired two-tailed Student's t-test.

FIGS. 4A-4J show representative data for systemic delivery of rAAV9.miR-214-3p TuD reverses osteoporosis in mice. (A) Diagram of the study and treatment methods. Sham or OVX surgery was performed on 12-week-old female mice, and four weeks later, a single dose of rAAV9 (5×10¹³ kg/vg) carrying ctrl or miR-214-3p TuD or miR-34a-5p was i.v. injected. Seven weeks after the injection, mice were injected with calcein/Alizarin red (AR) for dynamic histomorphometry analysis. (B) miR-214-3p or miR-34a-5p expression in the tibia and serum was measured by RT-PCR and normalized to U6 (n=5). (C, G) Femoral bone mass was assessed by microCT. Representative 3D reconstruction and relative quantification are displayed (n=5). (D, H) Representative images of calcein/AR labeling and relative histomorphometric quantification of bone formation rate (BFR)/bone surface (BS) and mineral apposition rate (MAR) are displayed. Arrows indicate the distance between calcein and AR labeling. (E, I) Representative images of TRAP-stained longitudinal sections of AAV-treated femurs. (F, J) Serum levels of CTX-I in AAV-treated mice were assessed by ELISA (n=5). Scale bars: C, G, 400 μm; D, E, H, I, 50 μm. Values represent mean±SD: * P<0.05; ** P<0.01; and *** P<0.001 by an unpaired two-tailed Student's t-test (B, D, F, H, J) and one-way ANOVA test (C, G).

FIGS. 5A-5H show representative data for systemic delivery of rAAV9 carrying miR-34a-5p reverses osteoporosis in mice. (A) Diagram of the study and treatment methods. 24-month-old male mice were i.v. injected with a single dose of rAAV9 (5×10¹³ kg/vg) carrying ctrl or miR-214-3p TuD or miR-34a-5p, and seven weeks later, mice were injected with calcein/AR for dynamic histomorphometry analysis. (B) miR-214-3p or miR-34a-5p expression in the tibia was measured by RT-PCR and normalized to U6 (n=5). (C, F) The femoral bone mass of rAAV-treated mice was assessed by microCT. 3D reconstruction (C, F, left) and relative quantification (C, F, right) are displayed (n=5). (D, G) Representative images of calcein/AR labeling and relative histomorphometric quantification of BFR/BS and MAR are displayed (n=5). (E, H) Scrum levels of CTX-I in AAV-treated mice were assessed by ELISA (n=5). Scale bars: D, G, 50 μm. Values represent mean±SD: * P<0.05; ** P<0.01; *** P<0.001; and **** P<0.0001 by an unpaired two-tailed Student's t-test.

FIGS. 6A-6F show representative data for AAV9 vectors carrying miR-214-3p TuD or miR-34a-5p in healthy mice (A) 10-week-old mice were i.v. injected with a single dose of rAAV9 (5×10¹³ kg/vg) carrying ctrl, miR-214-3p TuD, or miR-34a-5p and eight weeks later, expression of miR-214-3p (left) or miR-34a-5p (right) in the indicated tissues was assessed by RT-PCR (n=5). (B-E) Femoral bone mass of rAAV-treated mice was assessed by microCT, demonstrating that the AAV treatment had little to no effect on normal bone homeostasis. Representative images of 3D reconstruction (B, D) and relative quantification (C, E) are displayed (n=5). (F) Representative images of H&E-stained longitudinal sections of the indicated tissues of AAV-treated mice are displayed (n=5). Scale bars: B, D, 400 μm; F, 100 μm. Values represent mean±SD: ns, not significant; * P<0.05; ** P<0.01; and *** P<0.001 by an unpaired two-tailed Student's t-test (A, B, D).

FIGS. 7A-7B show representative data for expression of miR-214-3p and miR-34a-5p in osteoporotic bones. Expression of miR-214-3p (A) or miR-34a-5p (B) in the tibia of 16-week-old female mice with Sham or OVX surgery or 2.5-month-old (young) or 24-month-old (aged) male mice was assessed by RT-qPCR. Values represent mean±SD: * P<0.05; ** P<0.01; and *** P<0.001 by an unpaired two-tailed Student's t-test.

FIGS. 8A-8D show representative data for in vitro transduction efficiency of rAAV9 vectors in the osteoblast and osteoclast. (A, C) Mouse bone marrow-derived stromal cells (BMSCs) were incubated with rAAV9.egfp carrying ctrl, miR-214-3p or miR-34a-5p for two days and transduction efficiency was assessed by EGFP expression using fluorescence microscopy. (B, D) Two days after treatment with MCSF and RANKL, mouse bone marrow-derived monocytes (BMMs) were incubated with rAAV9.egfp carrying ctrl, miR-214-3p or miR-34a-5p for two days and transduction efficiency was assessed by EGFP expression using fluorescence microscopy. Scale bars: 400 μm.

FIG. 9 shows representative data for tissue distribution of systemically delivered rAAV9 vectors in mice. Two-month-old healthy mice were i.v. injected with a single dose of PBS or rAAV9 (5×10¹³ kg/vg) carrying ctrl, miR-214-3p, or miR-34a-5p, and two weeks later, EGFP expression in individual tissues was assessed by fluorescence microscopy. Scale bars: 400 μm.

FIGS. 10A-10C show representative data for AAV9 vectors carrying miR-214-3p TuD or miR-34a-5p on serum calcium levels and bone senescence in aged mice. 24-month-old male mice (n=5) were i.v. injected with a single dose of rAAV9 (5×10¹³ kg/vg) carrying ctrl or miR-214-3p TuD or miR-34a-5p, and eight weeks later, calcium levels in the serum were measured by calorimetric assay (A). mRNA levels of cell senescence marker genes, including p21, p53, and IL-6, were assessed by RT-PCR analysis and normalized to Gapdh (B, C). Values represent mean±SD: * P<0.05; ** P<0.01; and ns, not significant by an unpaired two-tailed Student's t-test.

FIGS. 11A-11C show representative data for AAV9 vectors carrying miR-214-3p TuD or miR-34a-5p in healthy mice. Ten-week-old healthy mice were i.v. injected with a single dose of rAAV9 (5×10¹³ kg/vg) carrying ctrl, miR-214-3p TuD, or miR-34a-5p and eight weeks later, tests for blood glucose levels (A) and complete blood count (CBC) (B, C) were performed in AAV-treated mice (n=5). Values represent mean±SD: ns, not significant by an unpaired two-tailed Student's t-test.

### DETAILED DESCRIPTION

Aspects of the disclosure relate to compositions and methods for modulating bone growth, for example by increasing osteogenesis and/or decreasing osteoclastogenesis. The disclosure is based, in part, on recombinant adeno-associated virus (rAAV) vectors encoding microRNAs or miRNA inhibitors that inhibit endogenous miR-214-3p and/or mediate overexpression of miR-34a-5p in osteoblasts (OBs) and osteoclasts (OCs). In some embodiments, compositions described by the disclosure are useful for treating certain bone diseases or disorders, such as osteoporosis.

### Isolated Nucleic Acids

Compositions and methods for delivering a transgene (e.g. an inhibitory RNA, such as an shRNA or miRNA, or a miRNA inhibitor, such as a TuD) to a subject are provided in the disclosure. The compositions typically comprise an isolated nucleic acid encoding a transgene capable of modulating bone metabolism. For example, in some embodiments, a transgene reduces expression or activity of a target miRNA, such as a target miRNA associated with inhibiting bone formation or growth, or promoting bone loss. In some embodiments, a transgene increases expression or activity of a target miRNA, such as a target miRNA associated with promoting bone formation or growth, or inhibiting bone loss.

“Bone metabolism” generally refers to a biological process involving bone formation and/or bone resorption. In some embodiments, bone metabolism involves the formation of new bone as produced by osteoblasts (OBs) and differentiated osteocytes, and/or mature bone tissue being resorbed by osteoclasts (OCs). OBs arise from the bone marrow derived mesenchymal cells that ultimately differentiate terminally into osteocytes. OB (and osteocyte) functions or activities include but are not limited to bone formation, bone mineralization, and regulation of OC activity. Decreased bone mass has been observed to result from inhibition of OB and/or osteocyte function or activity. Increased bone mass has been observed to result from increased OB and/or osteocyte function or activity. OCs arise from bone marrow-derived monocytes and in some embodiments have been observed to be controlled by signals from OBs. OC functions include bone resorption. In some embodiments, decreased bone mass has been observed to result from increased OC activity. In some embodiments, increased bone mass has been observed to result from inhibition of OC activity.

In some embodiments, an isolated nucleic acid or an rAAV as described by the disclosure comprises a transgene encoding miR-34a-5p. In some embodiments, an isolated nucleic acid or an rAAV as described by the disclosure comprises a transgene encoding miR-214-3p. In some embodiments, the miR-34a-5p or miR-214-3p is a mouse miRNA (e.g., is processed from a mouse miR-34a-5p or miR-214-3p pri-miRNA). In some embodiments, the miR-34a-5p or miR-214-3p is a human miRNA (e.g., is processed from a human miR-34a-5p or miR-214-3p pri-miRNA).

In some embodiments, an isolated nucleic acid encodes one or more (e.g., 1, 2, 3, 4, 5, 6, 7, 8, 9, 10, or more) inhibitory nucleic acids, for example dsRNA, siRNA, shRNA, miRNA, artificial microRNA (ami-RNA), etc.). Generally, an inhibitory nucleic acid specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of a gene encoding a gene product (e.g., a protein) associated with bone metabolism (e.g., RANKL, TGFβ-induced factor homeobox 2 (Tgif2), hypoxia-inducible factor-la (Hifla), etc.). As used herein “continuous bases” refers to two or more nucleotide bases that are covalently bound (e.g., by one or more phosphodiester bond, etc.) to each other (e.g. as part of a nucleic acid molecule). In some embodiments, the at least one inhibitory nucleic acid is about 50%, about 60% about 70% about 80% about 90%, about 95%, about 99% or about 100% identical to the two or more (e.g., 2, 3, 4, 5, 6, 7, 8, 9, 10, 11, 12, 13, 14, 15, 16, 17, 18, 19, 20, 21, 22, 23, 24, 25, or more) continuous nucleotide bases of a gene encoding a gene product (e.g., a protein) associated with bone metabolism (e.g., RANKL, Tgif2, Hifla, etc.).

A “microRNA” or “miRNA” is a small non-coding RNA molecule capable of mediating transcriptional or post-translational gene silencing. Typically, miRNA is transcribed as a hairpin or stem-loop (e.g., having a self-complementarity, single-stranded backbone) duplex structure, referred to as a primary miRNA (pri-miRNA), which is enzymatically processed (e.g., by Drosha, DGCR8, Pasha, etc.) into a pre-miRNA. The length of a pri-miRNA can vary. In some embodiments, a pri-miRNA ranges from about 100 to about 5000 base pairs (e.g., about 100, about 200, about 500, about 1000, about 1200, about 1500, about 1800, or about 2000 base pairs) in length. In some embodiments, a pri-miRNA is greater than 200 base pairs in length (e.g., 2500, 5000, 7000, 9000, or more base pairs in length.

Pre-miRNA, which is also characterized by a hairpin or stem-loop duplex structure, can also vary in length. In some embodiments, pre-miRNA ranges in size from about 40 base pairs in length to about 500 base pairs in length. In some embodiments, pre-miRNA ranges in size from about 50 to 100 base pairs in length. In some embodiments, pre-miRNA ranges in size from about 50 to about 90 base pairs in length (e.g., about 50, about 52, about 54, about 56, about 58, about 60, about 62, about 64, about 66, about 68, about 70, about 72, about 74, about 76, about 78, about 80, about 82, about 84, about 86, about 88, or about 90 base pairs in length).

Generally, pre-miRNA is exported into the cytoplasm, and enzymatically processed by Dicer to first produce an imperfect miRNA/miRNA* duplex and then a single-stranded mature miRNA molecule, which is subsequently loaded into the RNA-induced silencing complex (RISC). Typically, a mature miRNA molecule ranges in size from about 19 to about 30 base pairs in length. In some embodiments, a mature miRNA molecule is about 19, about 20, about 21, about 22, about 23, about 24, about 25, about 26, about 27, about 28, about 29, or 30 base pairs in length.

In some aspects, the disclosure provides isolated nucleic acids and vectors (e.g., rAAV vectors) that encode one or more artificial miRNAs. As used herein “artificial miRNA” or “amiRNA” refers to an endogenous pri-miRNA or pre-miRNA (e.g., a miRNA backbone, which is a precursor miRNA capable of producing a functional mature miRNA), in which the miRNA and miRNA* (e.g., passenger strand of the miRNA duplex) sequences have been replaced with corresponding amiRNA/amiRNA* sequences that direct highly efficient RNA silencing of the targeted gene, for example as described by Eamens et al. (2014), Methods Mol. Biol. 1062:211-224. For example, in some embodiments an artificial miRNA comprises a miR-155 pri-miRNA backbone into which a sequence encoding a bone metabolism modulating (e.g., bone formation inhibiting agent) miRNA has been inserted in place of the endogenous miR-155 mature miRNA-encoding sequence. In some embodiments, miRNA (e.g., an artificial miRNA) comprises a miR-155 backbone sequence, a miR-30 backbone sequence, a mir-64 backbone sequence, or a miR-122 backbone sequence.

In some embodiments, an artificial microRNA is between 6-50 nucleotides in length. In some embodiments, an artificial microRNA is between 8-24 nucleotides in length. In some embodiments, an artificial microRNA is between 12-36 nucleotides in length. In some embodiments, an artificial microRNA is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, a nucleic acid described herein encodes a miR-34a miRNA, which in humans is found on chromosome 1. In some embodiments, a miR-34a miRNA is described by miRBase accession number MIMAT0000255 or MIMAT0000542. In some embodiments, a nucleic acid (e.g., rAAV vector) described by the disclosure encodes a miR-34a-5p mature sequence, for example as set for in SEQ ID NO: 9 (5′-UGGCAGUGUCUUAGCUGGUUGU-3′). In some embodiments, miR-34a is encoded by a human pri-miRNA comprising the sequence: GGCCAGCUGUGAGUGUUUCUUUGGCAGUGUCUUAGCUGGUUGUUGUGAGCAAUA GUAAGGAAGCAAUCAGCAAGUAUACUGCCCUAGAAGUGCUGCACGUUGUGGGGC CC, as set forth in SEQ ID NO: 10. In some embodiments, miR-34a is encoded by a mouse pri-miRNA comprising the sequence: CCAGCUGUGAGUAAUUCUUUGGCAGUGUCUUAGCUGGUUGUUGUGAGUAUUAGC UAAGGAAGCAAUCAGCAAGUAUACUGCCCUAGAAGUGCUGCACAUUGU, as set forth in SEQ ID NO: 25.

In some embodiments, a nucleic acid described herein encodes a miR-214 miRNA, which in humans is found on chromosome 1. In some embodiments, a miR-214 miRNA is described by miRBase accession number MIMAT0000661 or MIMAT0000271. In some embodiments, a nucleic acid (e.g., rAAV vector) described by the disclosure encodes a miR-214-3p mature sequence, for example as set for in SEQ ID NO: 11 (5′-ACAGCAGGCACAGACAGGCAGU-3′). In some embodiments, miR-214 is encoded by a pri-miRNA comprising the sequence: GGCCUGGCUGGACAGAGUUGUCAUGUGUCUGCCUGUCUACACUUGCUGUGCAGA ACAUCCGCUCACCUGUACAGCAGGCACAGACAGGCAGUCACAUGACAACCCAGCC U, as set forth in SEQ ID NO: 12.

Aspects of the disclosure relate to miRNA inhibitors that reduce expression or activity of miRNAs associated with bone metabolism, for example miRNA-214 or miR-34a. As used herein, the term “miRNA Inhibitor” refers to an agent that blocks miRNA expression, processing and/or function. A variety of miRNA Inhibitor have been disclosed in the art. Non-limiting examples of miRNA inhibitors include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex. (See, e.g., Ebert, M. S. Nature Methods, Epub Aug. 12, 2007; Takeshi Haraguchi, et al., Nucleic Acids Research, 2009, Vol. 37, No. 6 e43, the contents of which relating to TuD RNAs are incorporated herein by reference).

Typically, a miRNA inhibitor is a nucleic acid molecule that comprises at least one miRNA binding site, e.g., an miR-214-3p binding site. The miRNA inhibitors may comprise 1 miRNA binding site, 2 miRNA binding sites, 3 miRNA binding sites, 4 miRNA binding sites, 5 miRNA binding sites, 6 miRNA binding sites, 7 miRNA binding sites, 8 miRNA binding sites, 9 miRNA binding sites, 10 miRNA binding sites, or more miRNA binding sites. As used herein, the term “miRNA binding site,” with reference to a miRNA inhibitor, refers to a sequence of nucleotides in a miRNA inhibitor that are sufficiently complementary with a sequence of nucleotides in a miRNA to effect base pairing between the miRNA inhibitor and the miRNA. Typically, a miRNA binding site comprises a sequence of nucleotides that are sufficiently complementary with a sequence of nucleotides in a miRNA to effect base pairing between the miRNA inhibitor and to thereby inhibit binding of the miRNA to a target mRNA.

As used herein the term “complementary” or “complementarity” refers to the ability of a nucleic acid to form hydrogen bond(s) with another RNA sequence by either traditional Watson-Crick or other non-traditional base pairing. In reference to the nucleic molecules of the present invention, the binding free energy for a nucleic acid molecule with its target or complementary sequence is sufficient to allow the relevant function of the nucleic acid to proceed, e.g., miRNA inhibition. Determination of binding free energies for nucleic acid molecules is well known in the art (see, e.g., Turner et al., 1987, CSH Symp. Quant. Biol. LII pp. 123 133; Frier et al., 1986, Proc. Nat. Acad. Sci. USA 83:9373 9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. In some embodiments the nucleic acids have 90%, 91%, 92%, 93%, 94%, 95%, 96%, 97%, 98%, 99% or 100% complementarity.

For a miRNA inhibitor having two miRNA binding sites, the first miRNA binding site and the second miRNA binding site may be complementary, e.g., at a sequence of 2 to 10 nucleotides in length. In one example, the first miRNA binding and the second miRNA binding site are complementary at a sequence of 4 nucleotides in length. Each miRNA binding site of a miRNA inhibitor may be any of a variety of lengths. For example, the miRNA binding site of a miRNA inhibitor may be 5 nucleotides to 35 nucleotides, 10 nucleotides to 30 nucleotides, or 15 nucleotides to 25 nucleotides. Typically, the length of the miRNA binding site depends on the length and/or structure of the miRNA to which it binds.

Often a miRNA binding site of a miRNA inhibitor of the invention is flanked by one or more stem sequence. As used herein the term “stem sequence” refers to a sequence of a nucleic acid that results in intramolecular base pairing. In some embodiments, stem sequences are not complementary with a target miRNA. Intramolecular base pairing may occur when two stem sequence regions of a miRNA inhibitor, usually palindromic sequences, base-pair to form a double helix, which may end in an unpaired loop. Thus, based pairing may form within a stem sequence or between two stem sequences. A stem sequence may be of a variety of lengths. For example, a stem sequence may be in range 3 nucleotides to 200 nucleotides, 3 nucleotides to 100 nucleotides, 3 nucleotides to 50, 3 nucleotides to 25 nucleotides, 10 nucleotides to 20 nucleotides, 20 nucleotides to 30 nucleotides, 30 nucleotides to 40 nucleotides, 40 nucleotides to 50 nucleotides, or 50 nucleotides to 100 nucleotides. A stem sequence may be up to 5 nucleotides, up to 10 nucleotides, up to 20 nucleotides, up to 50 nucleotides, up to 100 nucleotides, up to 200 nucleotides, or more. Linker sequences may also be included in a miRNA inhibitor. The miRNA inhibitor may comprise a first miRNA binding site and a second miRNA binding site, each binding site flanked by two stem sequences. A first stem sequence may flank the first miRNA binding site at its 5′-end, a second stem sequence may flank the first miRNA binding site at its 3′-end and the second miRNA binding site at its 5′-end, and a third stem sequence may flank the second miRNA binding site at its 3′-end. The skilled artisan will readily envision other configurations of binding sites and flanking stem sequences.

The miRNA binding site of a miRNA inhibitor of the invention may comprise a non-binding, central portion that is not complementary with the target miRNA (e.g., miR-214), flanked by two portions that are complementary with the target miRNA. A non-binding, central portion that is not complementary with the target miRNA need not be perfectly centered within the miRNA binding site. For example, a non-binding central portion may be flanked on either side by portions that are complementary with the target miRNA that are of different lengths. A miRNA inhibitor of the invention may comprise multiple miRNA binding sites that have a non-binding, central portion that is not complementary with the target miRNA. The non-binding, central portion of a miRNA binding site may have any of a variety of lengths. For example, a non-binding, central portion of a miRNA binding site may be in a range of 1 nucleotide to 20 nucleotides, 1 nucleotide to 10 nucleotides, 1 nucleotide to 5 nucleotides. The non-binding, central portion of a miRNA binding site may have a length in a range of 3 to 5 nucleotides. In one example, the non-binding, central portion of a miRNA binding site has a length of 4 nucleotides. The length of the non-binding, central portion will typically depend on the length of the miRNA binding site.

Often the non-binding, central portion of a first miRNA binding site is at least partially complementary with the non-binding, central portion of a second miRNA binding site of the inhibitor. Thus, two binding sites of an inhibitor may base pair (hybridize) with each other. The non-binding, central portion of a first miRNA binding site of an inhibitor may be complementary with the non-binding, central portion of a second miRNA binding site of an inhibitor at, for example, 2 nucleotides to 10 nucleotides, depending on the length of the binding site and the non-binding central portion. The non-binding, central portion of a first miRNA binding site of an inhibitor may be complementary with the non-binding, central portion of a second miRNA binding site at, for example, 2 nucleotides, 3 nucleotides, 4 nucleotides, 5 nucleotides, 10 nucleotides, or more nucleotides, typically depending on the length of the binding site and the non-binding central portion.

Some aspects of this disclosure provide miRNA inhibitors that target a plurality of miRNAs. In some embodiments, targeting a plurality of miRNAs circumvents the problem of inhibition of an individual miRNA being compensated for by related miRNAs. In some embodiments, the plurality of miRNAs belong to a family of miRNAs. In some embodiments, the plurality of miRNAs share at least some sequence identity. For example, in some embodiments, the plurality of miRNAs each comprise at least one stretch of 5 or more nucleotides that is identical across all of the plurality of miRNAs. In some embodiments, the plurality of miRNAs each comprise at least one stretch of 5 or more nucleotides that is at least 70%, at least 80%, at least 90%, at least 95%, or at least 98% identical to the consensus sequence of that stretch of nucleotides of the plurality of target miRNAs.

The term “consensus sequence,” as used herein, refers to a sequence of nucleotides that reflects the most common nucleotide shared by multiple nucleotide sequences at a specific position. In some embodiments, the multiple nucleotide sequences are related nucleotide sequences, for example, sequences of members of the same miRNA family. In some embodiments, a consensus sequence is obtained by aligning two or more sequences and determining the nucleotide most commonly found or most abundant in the aligned sequences at a particular position. Methods and algorithms for sequence alignment for obtaining consensus sequences from a plurality of sequences are well known to those of skill in the art and the invention is not limited in this respect.

In some embodiments, the miRNA inhibitor targeting a plurality of miRNAs is TuD comprising at least one miRNA binding site complementary to a consensus sequence of the plurality of miRNAs. In some embodiments, the consensus sequence is at least 5, at least 6, at least 7, at least 8, at least 9, at least 10, at least 11, at least 12, at least 13, at least 14, at least 15, at least 16, at least 17, at least 18, at least 19, or at least 20 nucleotides in length. In some embodiments, the miRNA inhibitor comprises a first miRNA binding site and a second miRNA binding site, wherein a first stem sequence flanks the first miRNA binding site at its 5′-end, a second stem sequence flanks the first miRNA binding site at its 3′-end and the second miRNA binding site at its 5′-end, and a third stem sequence flanks the second miRNA binding site at its 3′-end, wherein at least one of the miRNA binding sites comprises a nucleotide sequence complementary to a consensus sequence of the plurality of target miRNAs. In some embodiments, the first and the second miRNA binding sites are complementary to a consensus sequence of the plurality of target miRNAs. In some embodiments, the first and/or the second miRNA binding site is at least 7-%, at least 80%, at least 90%, at least 95%, or at least 98% complementary to a consensus sequence of the plurality of target miRNAs. In some embodiments, the consensus sequence the first miRNA binding site is complementary to is directly adjacent to the consensus sequence the second miRNA binding site is complementary to.

In some embodiments, a miRNA inhibitor is provided that targets a miR-214 (e.g., miR-214-3p) or miR-34a (e.g., miR-34a-5p). In some embodiments, the miRNA inhibitor comprises a sequence of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or 26, contiguous nucleotides of SEQ ID NO: 5.

In some embodiments, a nucleic acid encodes an miRNA inhibitor having at least one miRNA binding site, e.g., an miR-214-3p binding site. The miRNA inhibitors may comprise 1 miRNA binding site, 2 miRNA binding sites, 3 miRNA binding sites, 4 miRNA binding sites, 5 miRNA binding sites, 6 miRNA binding sites, 7 miRNA binding sites, 8 miRNA binding sites, 9 miRNA binding sites, 10 miRNA binding sites, or more miRNA binding sites. As used herein, the term “miRNA binding site,” with reference to a miRNA inhibitor, refers to a sequence of nucleotides in a miRNA inhibitor that are sufficiently complementary with a sequence of nucleotides in a miRNA to effect base pairing between the miRNA inhibitor and the miRNA.

Typically, a miRNA binding site comprises a sequence of nucleotides that are sufficiently complementary with a sequence of nucleotides in a miRNA to effect base pairing between the miRNA inhibitor and to thereby inhibit binding of the miRNA to a target mRNA.

In some embodiments, an inhibitory nucleic acid decreases expression or activity of a target gene (e.g., miR-214-3p) by between 50% and 99% (e.g., any integer between 50% and 99%, inclusive). In some aspects, an inhibitory nucleic acid decreases expression of a target gene by between 75% and 90%. In some aspects, an inhibitory nucleic acid decreases expression of a target gene by between 80% and 99%. In some embodiments, an inhibitory nucleic acid decreases expression of a SHN3 gene by between 50% and 99% (e.g., any integer between 50% and 99%, inclusive). In some embodiments, an inhibitory nucleic acid decreases expression of a SHN3 gene by between 75% and 90%. In some aspects, an inhibitory nucleic acid decreases expression of a SHN3 gene by between 80% and 99%.

In some embodiments, a miRNA inhibitor (e.g., TuD) increases expression or activity of a target gene (e.g., miR-34a-5p) by between 50% and 99% (e.g., any integer between 50% and 99%, inclusive). In some aspects, a miRNA inhibitor (e.g., TuD) increases expression or activity of a target gene (e.g., miR-34a-5p) by between 75% and 90%. In some aspects, a miRNA inhibitor (e.g., TuD) increases expression or activity of a target gene (e.g., miR-34a-5p) by between 80% and 99%. In some embodiments, a miRNA inhibitor (e.g., TuD) increases expression or activity of a target gene (e.g., miR-34a-5p) by between 50% and 99% (e.g., any integer between 50% and 99%, inclusive). In some embodiments, a miRNA inhibitor (e.g., TuD) increases expression or activity of a target gene (e.g., miR-34a-5p) by between 75% and 90%. In some aspects, a miRNA inhibitor (e.g., TuD) increases expression or activity of a target gene (e.g., miR-34a-5p) by between 80% and 99%.

In some embodiments, the transgene further comprises a nucleic acid sequence encoding one or more expression control sequences (e.g., a promoter, etc.). Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459). The cleavage activity of the 2A sequence has previously been demonstrated in artificial systems including plasmids and gene therapy vectors (AAV and retroviruses) (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459; de Felipe, P et al., Gene Therapy, 1999; 6:198-208; de Felipe, P et al., Human Gene Therapy, 2000; 11:1921-1931.; and Klump, H et al., Gene Therapy, 2001; 8:811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an enhanced chicken β-actin promoter. In some embodiments, a promoter is a U6 promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only. In some embodiments, an inducible promoter is induced (e.g., activated transcriptionally) by inflammation in the subject (e.g., the expression or release of inflammatory cytokines in the subject). In some embodiments, an inflammation-induced promoter comprises a NF-kappa B (NFκB) promoter. In some embodiments, a NF-kappa B (NFκB) promoter is a PB2 promoter.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, a tissue-specific promoter is a bone tissue-specific promoter. Examples of bone tissue-specific promoters include but are not limited to promoters of osterix, osteocalcin, type 1 collagen al, DMP1, cathepsin K, Rank, etc. In some embodiments, a promoter is an osteoblast-specific promoter. In some embodiments, an osteoblast-specific promoter comprises an osteocalcin (OCN) promoter. In some embodiments, a promoter is an osteoclast-specific promoter. In some embodiments, an osteoclast-specific promoter comprises a RANK promoter or NFκB promoter, such as a PB2 promoter.

Following systemic delivery, rAAV9 vectors can target additional tissues such as liver, lungs, heart, and skeletal muscle, which may cause adverse effects. The skilled artisan will appreciate that binding sites may be selected to control the expression of a transgene in a tissue specific manner. Therefore, the present disclosure provides tissue-specific, endogenous miRNAs to repress transgene expression in liver (e.g., miR-122) and/or lungs (e.g., miR-142), by engineering perfectly complementary miRNA-binding sites into the AAV vector genome. In some embodiments the rAAV comprises at least one tissue specific endogenous miRNA. In some embodiments, the tissue specific endogenous miRNA is a miR-122. In some embodiments, the tissue specific endogenous miRNA is a miR-142. The target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding an inhibitory RNA (e.g., miRNA), it may be desirable to drive expression of the protein coding region using a first promoter sequence (e.g., a first promoter sequence operably linked to the protein coding region), and to drive expression of the inhibitory RNA encoding region with a second promoter sequence (e.g., a second promoter sequence operably linked to the inhibitory RNA encoding region). Generally, the first promoter sequence and the second promoter sequence can be the same promoter sequence or different promoter sequences. In some embodiments, the first promoter sequence (e.g., the promoter driving expression of the protein coding region) is an RNA polymerase III (polIII) promoter sequence. Non-limiting examples of polIII promoter sequences include U6 and H1 promoter sequences. In some embodiments, the second promoter sequence (e.g., the promoter sequence driving expression of the inhibitory RNA) is an RNA polymerase II (polII) promoter sequence. Non-limiting examples of polII promoter sequences include T7, T3, SP6, RSV, and cytomegalovirus promoter sequences. In some embodiments, a polIII promoter sequence drives expression of an inhibitory RNA (e.g., miRNA) encoding region. In some embodiments, a polII promoter sequence drives expression of a protein coding region.

Recombinant AAVs (rAAVs)

The nucleic acids of the disclosure may be recombinant adeno-associated virus (AAV) vectors (rAAV vectors). In some embodiments, a nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more proteins and/or inhibitory nucleic acids (e.g., shRNA, miRNAs, etc.) comprising a nucleic acid that targets an endogenous mRNA of a subject. The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (Sec, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the nucleic acid (e.g., the rAAV vector) comprises at least one ITR having a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the nucleic acid comprises a region (e.g., a first region) encoding an AAV2 ITR.

In some embodiments, the nucleic acid further comprises a region (e.g., a second region, a third region, a fourth region, etc.) comprising a second AAV ITR. In some embodiments, the second AAV ITR has a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16 (10): 1648-1656.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, an anellovirus vector (e.g., Anellovirus vector as described in US20200188456A1), etc. Thus, the term includes cloning and expression vehicles, as well as viral vectors. In some embodiments, useful vectors are contemplated to be those vectors in which the nucleic acid segment to be transcribed is positioned under the transcriptional control of a promoter. In some embodiments, the vector is a plasmid. In some embodiments the vector is a Baculovirus vector.

As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle. The instant invention is based, in part, on the recognition that DNA fragments encoding RNA hairpin structures (e.g. shRNA, miRNA, and AmiRNA) can serve a function similar to a mutant inverted terminal repeat (mTR) during viral genome replication, generating self-complementary AAV vector genomes. For example, in some embodiments, the disclosure provides rAAV (e.g. self-complementary AAV; scAAV) vectors comprising a single-stranded self-complementary nucleic acid with inverted terminal repeats (ITRs) at each of two ends and a central portion comprising a promoter operably linked with a sequence encoding a hairpin-forming RNA (e.g., shRNA, miRNA, ami-RNA, etc.). In some embodiments, the sequence encoding a hairpin-forming RNA (e.g., shRNA, miRNA, ami-RNA, etc.) is substituted at a position of the self-complementary nucleic acid normally occupied by a mutant ITR.

“Recombinant AAV (rAAV) vectors” are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The instant disclosure provides a vector comprising a single, cis-acting wild-type ITR. In some embodiments, the ITR is a 5′ ITR. In some embodiments, the ITR is a 3′ ITR. Generally, ITR sequences are about 145 bp in length. Preferably, substantially the entire sequences encoding the ITR(s) is used in the molecule, although some degree of minor modification of these sequences is permissible. The ability to modify ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). For example, an ITR may be mutated at its terminal resolution site (TR), which inhibits replication at the vector terminus where the TR has been mutated and results in the formation of a self-complementary AAV. Another example of such a molecule employed in the present disclosure is a “cis-acting” plasmid containing the transgene, in which the selected transgene sequence and associated regulatory elements are flanked by the 5′ AAV ITR sequence and a 3′ hairpin-forming RNA sequence. AAV ITR sequences may be obtained from any known AAV, including presently identified mammalian AAV types. In some embodiments, an ITR sequence is an AAV2, AAV3, AAV4, AAV5, AAV6, AAV7, AAV8, AAVrh8, AAV9, AAV10, and/or AAVrh 10 ITR sequence.

In some embodiments, the rAAVs of the disclosure are pseudotyped rAAVs. For example, a pseudotyped AAV vector containing the ITRs of serotype X encapsidated with the proteins of Y will be designated as AAVX/Y (e.g. AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

Methods for obtaining recombinant AAVs having a desired capsid protein are well known in the art. (See, for example, US 2003/0138772), the contents of which are incorporated herein by reference in their entirety). Typically, the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125. In some embodiments, an AAV capsid protein is of a serotype derived from a non-human primate, for example scAAV.rh8, AAV.rh39, or AAV.rh43 serotype. In some embodiments, an AAV capsid protein is of an AAV9 serotype.

The disclosure is based, in part, on rAAVs comprising capsid proteins that have increased tropism for bone tissue. In some embodiments, the capsid proteins are grafted to a bone-targeting peptide. A heterologous bone-targeting peptide may target OCs (e.g., specifically, or preferentially targets OCs relative to OBs) or OBs (e.g., specifically, or preferentially targets OBs relative to OCs). In some embodiments, a bone-targeting peptide is an (AspSerSer)₆ peptide, which may also be referred to as a DSS6 peptide (e.g. SEQ ID NO: 13). Additional bone-targeting peptide is a HABP-19 peptide (CyEPRRyEVAyELyEPRRyEVAyEL; SEQ ID NO: 14), which may also be referred to as a HABP peptide. In some embodiments, a bone-targeting peptide is an (Asp) 8-14 (SEQ ID NO: 30) peptide comprising 8-14 aspartic acid residues. Further examples of bone-targeting peptides include but are not limited to those described by Ouyang et al. (2009) Lett. Organic Chem 6 (4): 272-277.

As used herein, “grafting” refers to joining or uniting of one molecule with another molecule. In some embodiments, the term grafting refers to joining or uniting of at least two molecules such that one of the at least two molecules is inserted within another of at least two molecules. In some embodiments, the term grafting refers to joining or uniting of at least two polymeric molecules such that one of at least two molecules is appended to another of at least two molecules. In some embodiments, the term grafting refers to joining or uniting of one polymeric molecule (e.g., a nucleic acid, a polypeptide) with another polymeric molecule (e.g., a nucleic acid, a polypeptide). In some embodiments, the term grafting refers to joining or uniting of at least two nucleic acid molecules such that one of at least two molecules is appended to another of at least two nucleic acid molecules.

In some embodiments, the term grafting refers to joining or uniting of at least two nucleic acid molecules such that one of the at least two nucleic acid molecules is inserted within another of the at least two nucleic acid molecules. For example, it has been observed that targeting peptides may be grafted to certain loci of a nucleic acid encoding a VP2 AAV capsid protein. In some embodiments, a targeting peptide (e.g. a bone-targeting peptide) is inserted at a position corresponding to the position between the codons encoding Q588 and A589 and/or N587 and R588 of an AAV2 or AAV9 VP2 capsid protein. In some embodiments, a targeting peptide is inserted at a position between the codons encoding N587 and R588 of an VP3 capsid protein (or a position corresponding to such amino acid positions in AAV2 or AAV9). In some embodiments, a targeting peptide is inserted at a position between the codons encoding S452 and G453 of an VP1 capsid protein. Other potential positions may be N587 and R588.

In some embodiments, a nucleic acid formed through grafting (a grafted nucleic acid) encodes a chimeric protein. In some embodiments, a grafted nucleic acid encodes a chimeric protein, such that one polypeptide is effectively inserted into another polypeptide (e.g. not directly conjugated before the N-terminus or after the C-terminus), thereby creating a contiguous fusion of two polypeptides. In some embodiments, a grafted nucleic acid encodes a chimeric protein, such that one polypeptide is effectively appended to another polypeptide (e.g. directly conjugated before the N-terminus or after the C-terminus), thereby creating a contiguous fusion of two polypeptides. In some embodiments, the term grafting refers to joining or uniting of at least two polypeptides, or fragments thereof, such that one of the at least two polypeptides or fragments thereof is inserted within another of the at least two polypeptides or fragments thereof. In some embodiments, the term grafting refers to joining or uniting of at least two polypeptides or fragments thereof such that one of the at least two polypeptides or fragments thereof is appended to another of the at least two polypeptides or fragments thereof.

In some embodiments, the disclosure relates to an adeno-associated virus (AAV) capsid protein that is conjugated to one or more bone-targeting moieties. A “bone-targeting moiety” generally refers to a small molecule, peptide, nucleic acid, etc., that facilitates trafficking of an rAAV to bone or bone tissue. For example, in some embodiments, a bone-targeting moiety is a peptide or small molecule that binds to a receptor on a bone cell (e.g., OB, OC, osteocyte, etc.). Examples of bone-targeting moieties include but are not limited to alendronate (ALE), polypeptides such as cyclic arginine-glycine-aspartic acid-tyrosine-lysine (cRGCyk) (SEQ ID NO: 31), Asp-Asp-Asp-Asp-Asp-Asp-Asp-Asp (D-Asp8) (SEQ ID NO: 32), and aptamers such as CH6. A bone-targeting moiety may be conjugated directly to a capsid protein or conjugated to a capsid protein via a linker molecule (e.g., an amino acid linker, a PEG linker, etc.).

In some embodiments, a linker is a glycine-rich linker. In some embodiments, a linker comprises at least two glycine residues. In some embodiments, a linker comprises GGGGS (SEQ ID NO: 33). In some embodiments, the linker comprises a formula selected from the group consisting of: [G]ₙ, [G]ₙS, [GS]ₙ, and [GGSG]ₙ, (SEQ ID NO: 34), wherein G is glycine and wherein n is an integer greater than one (e.g., 2, 3, 4, 5, 6, 7, 8, 9, 10, 11, 12, 13, 14, 15, 16, 17, 18, 19, 20, 21, 22, 23, 24, 25 or more). In some embodiments, n is an integer in a range of 2 to 10, 2 to 20, 5 to 10, 5 to 15, or 5 to 25. Accordingly, in some embodiments, a heterologous targeting peptide is conjugated to a linker.

In some embodiments, a capsid protein comprises one or more azide-bearing unnatural amino acids which are capable of reacting with an ADIBO-tagged bone-targeting moiety (e.g., via “click chemistry” to form a capsid protein-bone-targeting moiety conjugate. Capsid proteins comprising unnatural azide-bearing amino acids are described, for example by Zhang et al. (2016) Biomaterials 80:134-145, and use of ADIBO-based click chemistry for peptide conjugation is described, for example by Prim et al. (2013) Molecules 18 (8): 9833-49.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. Sec, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. Sec, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. In some embodiments, a host cell is a bacterial cell, yeast cell, insect cell (Sf9), or a mammalian (e.g., human, rodent, non-human primate, etc.) cell. In some embodiments, the host cell is a HEK293 cell. In some embodiments, the host cell is a SF9 cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

In some aspects, the present disclosure provides a recombinant AAV comprising a capsid protein and an isolated nucleic acid comprising a first region encoding an AAV ITR and a second region comprising a transgene, wherein the transgene encodes an artificial microRNA. The artificial microRNA may decrease the expression of a target gene in a cell (e.g. osteoblasts, osteoclasts, osteocytes, chondrocytes) or a subject. In some embodiments, the rAAV comprises an artificial microRNA that decreases the expression of SHN3 in a cell or a subject.

The rAAV may comprise at least one modification which increases targeting of the rAAV to bone cells (e.g., osteoblasts, osteoclasts, osteocytes, chondrocytes). Non-limiting examples of modifications which increase targeting of the rAAV to bone cells include heterologous bone-targeting peptides, AAV capsid serotypes (e.g., AAV1, AAV4, AAV5, AAV6, AAV6.2, AAV7, AAV8, AAV9, AAV10, AAVrh39, AAVrh43).

The foregoing methods for packaging recombinant vectors in desired AAV capsids to produce the rAAVs of the disclosure are not meant to be limiting and other suitable methods will be apparent to the skilled artisan.

### Modes of Administration and Compositions

The rAAVs of the disclosure may be delivered to a subject in compositions according to any appropriate methods known in the art. For example, an rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the bone (e.g., bone tissue) of a subject. By “bone tissue” is meant all cells and tissue of the bone and/or joint (e.g., cartilage, axial and appendicular bone, etc.) of a vertebrate. Thus, the term includes, but is not limited to, osteoblasts, osteocytes, osteoclasts, chondrocytes, and the like. Recombinant AAVs may be delivered directly to the bone by injection into, e.g., directly into the bone, via intrasynovial injection, knee injection, femoral intramedullary injection, etc., with a needle, catheter or related device, using surgical techniques known in the art. In some embodiments, rAAV as described in the disclosure are administered by intravenous injection. In some embodiments, the rAAV are administered by intramuscular injection.

Aspects of the instant disclosure relate to compositions comprising a recombinant AAV comprising a capsid protein and a nucleic acid encoding a transgene, wherein the transgene comprises a nucleic acid sequence encoding one or more bone metabolism modulating agents. In some embodiments, the nucleic acid further comprises one or more AAV ITRs. In some embodiments, the rAAV comprises an rAAV vector comprising the sequence set forth in any one of SEQ ID NO: 4, 6, 7, or 8 (or the complementary sequence thereof), or a portion thereof. In some embodiments, a composition further comprises a pharmaceutically acceptable carrier. In some embodiments, an rAAV vector encoding one or more miRNAs and/or one or more miRNA binding sites (e.g., a TuD) does not comprise a reporter protein (e.g., nucleic acid sequence encoding luciferase, EGFP, etc.). In some embodiments, an rAAV vector lacks protein coding nucleic acid sequences.

In some embodiments, compositions comprise a recombinant AAV comprising a capsid protein and a nucleic acid comprising a first region encoding an AAV ITR and a second region comprising a transgene, wherein the transgene encodes a miR-34a-5p. In some embodiments, the recombinant AAV comprises a sequence as set forth in SEQ ID NO: 4. In some embodiments, the capsid protein is an AAV9 capsid protein. In some embodiments, the capsid protein further comprises a heterologous bone-targeting peptide.

In some embodiments, compositions comprise a recombinant AAV comprising a capsid protein and a nucleic acid comprising a first region encoding an AAV ITR and a second region comprising a transgene, wherein the transgene encodes a miRNA inhibitor that inhibits miR-214-3p. In some embodiments, the recombinant AAV comprises a sequence as set forth in SEQ ID NO: 5 or 6. In some embodiments, the capsid protein is an AAV9 capsid protein. In some embodiments, the capsid protein further comprises a heterologous bone-targeting peptide.

Aspects of the disclosure provide a method of decreasing miR-214-3p expression or activity in a cell. A cell may be a single cell or a population of cells (e.g., culture). A cell may be in vivo (e.g., in a subject) or in vitro (e.g., in culture). A subject may be a mammal, optionally a human, a mouse, a rat, a non-human primate, a pig, a dog, a cat, a chicken, or a cow.

Expression or activity of miR-214-3p in a cell or subject may be decreased by between 50% and 99% (e.g., any integer between 50% and 99%, inclusive) using isolated nucleic acids, rAAVs, or compositions of the present disclosure. Expression or activity of miR-214-3p in a cell or subject may be decreased by between 75% and 90% using isolated nucleic acids, rAAVs, or compositions of the present disclosure. Expression or activity of miR-214-3p in a cell or subject may be decreased by between 80% and 99% using isolated nucleic acids, rAAVs, or compositions of the present disclosure.

Aspects of the disclosure provide a method of increasing miR-34a-5p expression or activity in a cell. A cell may be a single cell or a population of cells (e.g., culture). A cell may be in vivo (e.g., in a subject) or in vitro (e.g., in culture). A subject may be a mammal, optionally a human, a mouse, a rat, a non-human primate, a pig, a dog, a cat, a chicken, or a cow.

Expression or activity of miR-34a-5p in a cell or subject may be increased by between 50% and 99% (e.g., any integer between 50% and 99%, inclusive) using isolated nucleic acids, rAAVs, or compositions of the present disclosure. Expression or activity of miR-34a-5p in a cell or subject may be increased by between 75% and 90% using isolated nucleic acids, rAAVs, or compositions of the present disclosure. Expression or activity of miR-34a-5p in a cell or subject may be increased by between 80% and 99% using isolated nucleic acids, rAAVs, or compositions of the present disclosure.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An “effective amount” of an rAAV is an amount sufficient to target infect an animal, target a desired tissue (e.g., bone tissue). The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 109 to 1016 genome copies. In some cases, a dosage between about 1011 to 10¹³ rAAV genome copies is appropriate. In certain embodiments, 10¹² or 10¹³ rAAV genome copies is effective to target bone tissue.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, femoral intramedullary, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Therapeutic Methods

Methods for delivering an effective amount of a transgene (e.g., a nucleic acid or rAAV encoding a miR-34a-5p or miRNA inhibitor targeting miR-214-3p) to a subject are provided by the disclosure. In some embodiments, the methods comprise the step of administering to a subject an effective amount of an isolated nucleic acid encoding an interfering RNA capable of inhibiting bone loss (e.g., bone loss due to bone fracture, osteoporosis). In some embodiments, the methods comprise the step of administering to a subject an effective amount of an isolated nucleic acid encoding an interfering RNA capable of reversing bone loss. Thus, in some embodiments, isolated nucleic acids, rAAVs, and compositions described herein are useful for treating a subject having or suspected of having a disease or disorder associated with bone loss. As used herein, a “disease or disorder associated with dysregulated bone metabolism” refers to a condition characterized by an imbalance between bone deposition and bone resorption resulting in either 1) abnormally increased bone deposition (e.g., formation) relative to a healthy individual (e.g., a subject not having a disease characterized by imbalance between bone deposition and bone resorption), or 2) abnormally decreased bone deposition (e.g., formation) relative to a healthy individual (e.g., a subject not having a disease characterized by an imbalance between bone deposition and bone resorption), or 3) abnormally increased bone resorption (e.g., breakdown) relative to a healthy individual (e.g., a subject not having a disease characterized by imbalance between bone deposition and bone resorption), or 4) abnormally decreased bone resorption (e.g., breakdown) relative to a healthy individual (e.g., a subject not having a disease characterized by imbalance between bone deposition and bone resorption).

A “disease associated with reduced bone density” refers to a condition characterized by increased bone porosity resulting from either 1) abnormally decreased bone deposition (e.g., formation) relative to a healthy individual (e.g., a subject not having a disease characterized by decreased bone density), or 2) abnormally increased bone resorption (e.g., breakdown) relative to a healthy individual (e.g., a subject not having a disease characterized by decreased bone density). A disease associated with increased bone porosity may arise from either 1) abnormally decreased OB and/or osteocyte differentiation, function, or activity relative to a healthy individual (e.g., a subject not having a disease characterized by decreased bone density) and/or 2) abnormally increased OC differentiation, function, or activity relative to a healthy individual (e.g., a subject not having a disease characterized by decreased bone density). “Porosity” generally refers to the volume of fraction of bone not occupied by bone tissue.

A “disease associated with increased bone density” refers to a condition characterized by decreased bone porosity resulting from either 1) abnormally increased bone deposition (e.g., formation) relative to a healthy individual (e.g., a subject not having a disease characterized by increased bone density), or 2) abnormally decreased bone resorption (e.g., breakdown) relative to a healthy individual (e.g., a subject not having a disease characterized by increased bone density). A disease associated with decreased bone porosity may arise from either 1) abnormally increased OB and/or osteocyte differentiation, function, or activity relative to a healthy individual (e.g., a subject not having a disease characterized by increased bone density) and/or 2) abnormally decreased OC differentiation, function, or activity relative to a healthy individual (e.g., a subject not having a disease characterized by increased bone density).

Aspects of the present disclosure provide methods of treating a disease or disorder associated with dysregulated bone metabolism. Dysregulated bone metabolism may be diseases associated with reduced bone density (e.g., osteoporosis, critical sized-bone defects, a mechanical disorder resulting from disuse or injury). Dysregulated bone metabolism may be diseases associated with increased bone density (e.g., osteopetrosis, pycnodysostosis, sclerosteosis, acromegaly, fluorosis, myelofibrosis, hepatitis C-associated osteosclerosis, heterotrophic ossification).

In some embodiments, a subject having a disease or disorder associated with dysregulated bone metabolism has one or more signs or symptoms of an inflammatory disease. Examples of inflammatory diseases include but are not limited to rheumatoid arthritis (RA), psoriasis, ankylosing spondylitis, systemic lupus erythematosus, multiple sclerosis, inflammatory bowel diseases, periodontitis, and pemphigus vulgaris. In some embodiments, a subject having an inflammatory disease is characterized as having an increased level or amount of inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) or other markers of inflammation, relative to a normal, healthy subject. In some embodiment, the subject having an inflammatory disease has the level or amount of inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) or other markers of inflammation increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a normal, healthy subject.

In some embodiments, administering the nucleic acid, the rAAV, the vector, the bone graft substitute improves bone formation and/or bone healing in a subject by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the nucleic acid, the rAAV, the vector, the bone graft substitute stimulate bone regeneration and/or reversing bone loss in a subject by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

As used herein, the improvement or stimulation is relative to a control. The control can be in a state that is prior to the administration of the isolated nucleic acid, the rAAV, the vector, and the bone graft substitute. The improvement or stimulation is relative to a subject that has not been administered the isolated nucleic acid, the rAAV, the vector, and the bone graft substitute.

As used herein, a “normal, healthy subject” refers to a subject who does not have, is not suspected of, or is at risk of developing a disease or disorder. In some embodiments, the disease or disorder is an inflammatory disease. In some embodiments, the disease or disorder is associated with bone metabolism. In some embodiments, a normal, healthy subject can be a control described herein.

As used herein, the term “treating” refers to the application or administration of a composition, isolated nucleic acid, vector, or rAAV as described herein to a subject having bone loss or a predisposition toward a bone loss condition, with the purpose to cure, heal, alleviate, relieve, alter, remedy, ameliorate, improve, or affect the disorder, the symptom of the disease, or the predisposition toward the inflammatory condition.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of inflammatory diseases includes initial onset and/or recurrence.

In some embodiments, methods of treating osteoporosis comprise administering to a subject in need thereof a recombinant AAV (rAAV) comprising a transgene. A rAAV may comprise a modification that promotes its targeting to bone cells (e.g., osteoclasts and osteoblasts). Non-limiting modifications of rAAVs that promote its targeting to bone cells include modification of capsid proteins with heterologous bone-targeting peptides, modification of rAAV vectors with bone-specific promoters, and use of AAV serotypes with increased targeting to bone relative to other tissues.

In some embodiments, an “effective amount” or “amount effective of a substance” in the context of a composition or dose for administration to a subject refers to an amount sufficient to produce one or more desired effects (e.g., to preserve bone tissue or reverse bone loss). In some embodiments, an effective amount of a nucleic acid is an amount sufficient to transfect (or infect in the context of rAAV-mediated delivery) a sufficient number of target cells of a target tissue of a subject. In some embodiments, a target tissue is bone tissue (e.g., bone and bone tissue cells, such as OBs, OCs, osteocytes, chondrocytes, etc.). In some embodiments, an effective amount of a nucleic acid (e.g., which may be delivered via an rAAV) may be an amount sufficient to have a therapeutic benefit in a subject, e.g., to increase activity or function of OBs and/or osteocytes, to inhibit activity of OBs and/or osteocytes, to increase activity of function of OCs, to inhibit activity or function of OCs, etc. In some embodiments, an effective amount of an isolated nucleic acid disclosed herein may partially or fully rescue bone losses. In some embodiments, an effective amount of an isolated nucleic acid disclosed herein may partially or fully alleviate the effects of the genes that cause bone losses. An effective amount can also involve delaying the occurrence of an undesired response. The effective amount will depend on a variety of factors such as, for example, the species, age, weight, health of the subject, the severity of a condition, the tissue to be targeted, the specific route of administration and like factors, and may thus vary among subject and tissue as described elsewhere in the disclosure.

Exemplary embodiments of the invention will be described in more detail by the following examples. These embodiments are exemplary of the invention, which one skilled in the art will recognize is not limited to the exemplary embodiments.

### Example

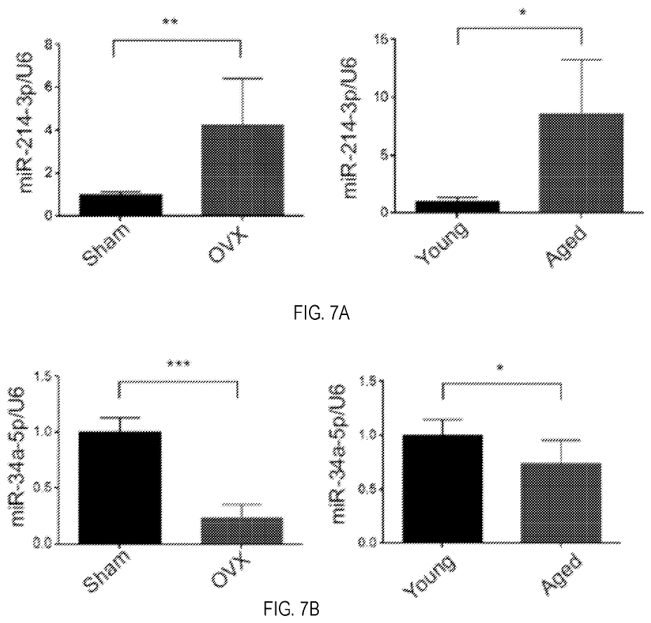

It was observed that miR-214-3p levels in the bone were highly upregulated by aging-associated or postmenopausal osteoporosis, while miR-34a-5p levels were downregulated in the same conditions (e.g., as shown in FIGS. 7A-7B), indicating their association with osteoporosis. miR-214 has been observed to directly target activating transcription factor 4 (ATF4) to inhibit osteoblast differentiation, and also to target phosphatase and tensin homolog (Pten) and upregulate the phosphatidylinositol 3-kinase (PI-3K) pathway to promote osteoclast differentiation. Additionally, exosomal miR-214-3p produced by osteoclasts has been observed to function as a coupling factor that suppresses the differentiation of neighboring osteoblasts. Conversely, miR-34a-5p plays a negative role in osteoclast differentiation by targeting RANKL, TGFβ-induced factor homeobox 2 (Tgif2), and hypoxia-inducible factor-la (Hifla), which downregulates the OPG/RANK/RANKL and IL-33/Notch1 signaling. On the other hand, elevated levels of miR-34a-5p also promote osteogenic differentiation of mesenchymal stromal cells (MSCs) upon X-ray irradiation or artesunate treatment. Hence, miR-214-3p or miR-34a-5p that can regulate both osteoblast and osteoclast differentiation are attractive targets for osteoporosis therapy.

This example describes a novel osteoporosis therapy using rAAV9-mediated modulation of two osteoblast/osteoclast-regulating miRNAs, miR-214-3p and miR-34a-5p. rAAV9-mediated inhibition of endogenous miR-214-3p or overexpression of miR-34a-5p counteracted bone loss in mouse models for aging-associated and postmenopausal osteoporosis by simultaneously promoting osteoblast-mediated bone formation while inhibiting osteoclast-mediated bone resorption. Conversely, overexpression of miR-214-3p or inhibition of miR-34a-5p in healthy mice resulted in low bone mass, resembling osteoporotic mice with upregulation of miR-214-3p and downregulation of miR-34a-5p. Little to no apparent adverse effects were observed in non-skeletal tissues of AAV-treated mice.

### AAV Vector Design and Production

Anti-miR-34a-5p TuD and anti-miR-214-3P TuD were designed and incorporated into pAAVsc.CB6-Gluc vector plasmid as previously described. gBlocks containing pre-miR-34a or pre-miR-214 and its˜100 base pairs flanking sequences at both ends were cloned into the intron of pAAVsc.CB6-EGFP vector plasmid. To monitor the miRNA activity, sensor plasmids were made by inserting three copies of miRNA binding sites after the β-galactosidase reporter gene in the pmiCHECK plasmid. The sequences of oligos and gBlocks for plasmid construction are shown in Table 1. rAAV9 was produced by transient HEK 293 cell transfection and CsCl sedimentation. Vector preparations were determined by ddPCR, and purity was assessed by 4%-12% SDS-acrylamide gel electrophoresis and silver staining (Invitrogen). AAV vector genomes expressing miR-214 and miR-34a were transiently transfected into HEK293T cells, and 48 hours later, transfection efficiency was examined by EGFP expression using fluorescence microscopy. Additionally, miRNA expression was examined by RT-PCR using the TaqMan miRNA assay kit (Applied Biosystems). For functional validation, the AAV vector genomes (500 ng) expressing control, miR-214, or miR-34a were transfected into HEK293T cells along with the sensor plasmids (100 ng) for control (pmiCheck-Scr), miR-214-3p (pmiCheck-miR-214-3p), or miR-34a-5p (pmiCheck-miR-34a-5p). After 48 hours, β-galactosidase activity was measured by using Galacto-Star™ β-Galactosidase Reporter Gene Assay System for Mammalian Cells (Applied Biosystems, T10¹²) and normalized to firefly luciferase activity (Promega) according to the manufacturer's protocol.

AAV vector genomes expressing miR-399 214-3p TuD or miR-34a-5p TuD were transfected into HEK293T cells at different concentrations (5 ng, 25 ng, 250 ng and 500 ng) along with the sensor plasmids (100 ng) for control (pmiCheck-Scr), miR-214-3p (pmiCheck-miR-214-3p), or miR-34a-5p (pmiCheck-miR-34a-5p) in presence of AAV vector genomes expressing miR-214-3p or miR-34a-5p, respectively. After 48 hours, β-galactosidase activity was measured and normalized to firefly luciferase activity.

### Cell Culture

HEK293T cells were procured from American Type Culture Collection (ATCC) (Rockville, MD). The cells were grown in DMEM (Corning) supplemented with 10% FBS (Corning), 2 mM L-glutamine (Corning), 1% penicillin/streptomycin (Corning) and 1% nonessential amino acids (Corning) at 37° C. under a humidified atmosphere of 5% CO₂.

### Osteoblast and Osteoclast Culture

For osteoblast culture, bone marrow stromal cells (BMSCs) were harvested from the long bones of 8-week-old wild-type mice (C57BL/6J, Jackson laboratory). Briefly, femurs and tibias were crushed and then, filtered through 70 μm cell strainer. After treatment with red blood cell lysis buffer, cells were suspended with α-minimal essential medium (α-MEM) containing 10% FCS and 1% penicillin/streptomycin (Corning) and cultured in the 60 mm plate at a density of 1×10⁷ cells/mL. All non-adherent cells were removed three days after the culture. Ascorbic acid (200 μM, Sigma, A8960) and β-glycerophosphate (10 mM, Sigma, G9422) were added to differentiate BMSCs into osteoblasts. BMSCs were seeded at a concentration of 1×10⁴ cells/well in a 24-well plate, and one day later, cells were incubated with rAAV9 vectors (5×10¹² GC) for two days. To detect osteoblast differentiation, alkaline phosphatase 422 (ALP) activity was determined. Briefly, differentiated osteoblasts were washed with PBS and incubated with a solution containing 6.5 mM Na₂CO₃, 18.5 mM NaHCO₃, 2 mM MgCl₂, and phosphatase substrate (Sigma, S0942), and ALP activity was measured by spectrometer. Alternatively, ALP staining was performed using Fast Blue (Sigma, FBS25) and Naphthol AS-MX (Sigma, 855) after fixation in 10% neutral formalin buffer. For the osteoclast differentiation assay, mouse bone marrow monocytes (BMMs) were harvested from bone marrow cells in the long bones of 8-week-old wild-type mice (C57BL/6J, Jackson Laboratory). Briefly, bone marrow cells were flushed out from the femurs and tibias, treated with red blood cell lysis buffer, and suspended with 10% FCS and 1% penicillin/streptomycin (Corning). Cells were cultured in the presence of M-CSF (10 ng/ml, R&D Systems, 416-ML) and one day later, non-adherent cells were plated at a density of 0.5×10⁶ cells/well in 24-well plates. BMMs were differentiated into osteoclasts by treating with M-CSF (10 ng/ml) and RANKL (20 ng/ml, R&D Systems, 462-TEC) and two days later, incubated with rAAV9 vectors (5×10¹² GC) under osteoclast differentiation conditions for two days. To assess osteoclast differentiation, TRAP (tartrate-resistant acid phosphatase) staining was performed using a leukocyte acid phosphatase staining kit (Sigma) according to the manufacturer's protocol. The TRAP-stained osteoclasts were detected by Evos microscope (Applied Biosystems).

### Quantitative PCR

A TaqMan microRNA assay kit (Applied Biosystems) was used to measure the expression of miR-214-3p and miR-34a-5p. miRNAs were isolated from tibias, osteoblasts, or osteoclasts using the mirVana miRNA isolation kit (Ambion), followed by cDNA synthesis using the TaqMan miRNA reverse transcription kit (Applied Biosystems). The cDNA was used for RT447 PCR using a TaqMan miRNA assay kit (Applied Biosystems) according to the manufacturer's protocol: miR-214-3p (assay id: 002306), miR-34a-5p (assay id: 000426), U6 (assay id: 001973, internal control). Alternatively, total RNA was extracted using QIAzol (QIAGEN), followed by cDNA synthesis using the high-capacity cDNA reverse transcription kit (Applied Biosystems). RT-PCR analysis was performed using SYBR Green PCR master mix (Bio-Rad) with a CFX Connect RT-PCR detection system (Bio-Rad).

Measurement of cross-linked C-telopeptide of type I collagen (CTx-I) and Serum Calcium

Wild-type serums were harvested from AAV-treated mice by heart puncture after euthanasia and assessed by CTx-I ELISA assay (ABclonal, MC0850). Calcium levels in the serum were assessed by calcium calorimetric assay kit (Sigma, MAK022) as per manufacturer's protocol

Delivery of rAAV9 Vectors

10-week-old wild-type female mice were randomly divided into five groups and injected i.v. via tail vein with a single dose of rAAV9 vectors (5×10¹³ kg/vg, 200 μL) carrying control, miR-214-3p, miR-214-3p TuD, miR-34a-5p, or miR34a-5p TuD. Eight weeks later, miRNA expression, skeletal analysis, and histopathology were performed on tibias. For the postmenopausal osteoporosis study, 12-week-old female mice were anesthetized and bilaterally ovariectomized (OVX) or sham operated. OVX mice were randomly assigned, and four weeks later, mice were i.v. injected with a single dose of rAAV9 vectors (5×10¹³ kg/vg, 200 μL) carrying ctrl, miR-214-3p TuD, or miR-34a-5p. Sham mice were i.v. injected with rAAV9 carrying ctrl. Eight weeks later, mice were euthanized, and bone samples were harvested for RT-PCR, microCT, histology, and histomorphometry. For the senile osteoporosis study, 24-month-old female mice were i.v. injected with a single dose of rAAV9 vectors (5×10¹³ kg/vg, 200 μL) carrying ctrl, miR-214-3p TuD, or miR-34a-5p. Eight weeks later, mice were euthanized, and bone samples were harvested for RT-PCR, microCT, histology, and histomorphometry.

### MicroCT and Histology

MicroCT was used for qualitative and quantitative assessment of trabecular and cortical bone microarchitecture and performed by an investigator blinded to the genotypes of the animals under analysis. Briefly, femurs dissected from the indicated mice groups were scanned using a microCT (Scanco Medical) with a spatial resolution of 7 μm. For trabecular bone analysis of the distal femur, an upper 2.1 mm region beginning 280 μm proximal to the growth plate was contoured. Three-dimensional reconstruction images were obtained from contoured two-dimensional images by methods based on distance transformation of the binarized images. For cortical bone analysis of the femur, a mid-shaft region of 0.6 mm in length was used. All images presented are representative of the respective genotypes (n=5). Histological and histomorphometric analysis was carried out. For histological studies, femurs were dissected from AAV-treated mice, fixed in 10% neutral buffered formalin for 2 days, followed by decalcification for 2-4 weeks using 0.5 M tetrasodium EDTA. Further, tissues were dehydrated by passage through an ethanol series, cleared twice in xylene, embedded in paraffin, and sectioned at a thickness of 6 μm along the coronal plate from anterior to posterior. Decalcified femoral sections were stained with TRAP. For dynamic histomorphometric analysis, mice groups were subcutaneously injected at 6-day intervals with 25 mg/kg calcein (Sigma, C0875) and 50 mg/kg alizarin-3-methyliminodiacetic acid (Sigma, A3882) dissolved in 2% sodium bicarbonate solution. The distances between bone surfaces labeled by calcein (existing bone) and alizarin-3-methyliminodiacetic acid (newly formed bone) were used to assess MARs and mineralized surface/BS to calculate BFRs. After 2 days of fixation in 10% neutral buffered formalin, undecalcified femur samples were embedded in methyl methacrylate, and the proximal metaphyses of femurs were sectioned longitudinally (5 μm) and stained with TRAP for osteoclasts. A region of interest was defined in the trabecular bone of the metaphysis, and BFR/BS and MAR were measured using a Nikon Optiphot 2 microscope interfaced with a semiautomatic analysis system (OsteoMetrics). Measurements were taken on two sections/sample (separated by˜25 μm) and summed prior to normalization to obtain a single measure/sample in accordance with the American Society of Bone and Mineral Research Histomorphometry Nomenclature Committee.

### Blood Glucose and Complete Blood Cell Count

Whole-blood glucose levels in mice were measured using a hand-held whole-blood glucose meter (Mckesson) and corresponding glucose test strips. A blood drop was taken by snipping the tip of the tail with sharp scissors, and glucose levels were detected according to the manufacturer's protocol. CBC tests were performed to evaluate cellular components in the blood of AAV-treated mice, including white blood cells (WBCs), red blood cells (RBCs), lymphocytes, monocytes, hemoglobin, and platelets (PLTs). Blood drops were collected into a microtainer EDTA tube and tested within one hour at room temperature using an automated hematology analyzer (VetScan HM5, Zoetis, USA).

rAAV9-Mediated Modulation of miR-214-3p Regulates Osteoblast and Osteoclast Differentiation In Vitro

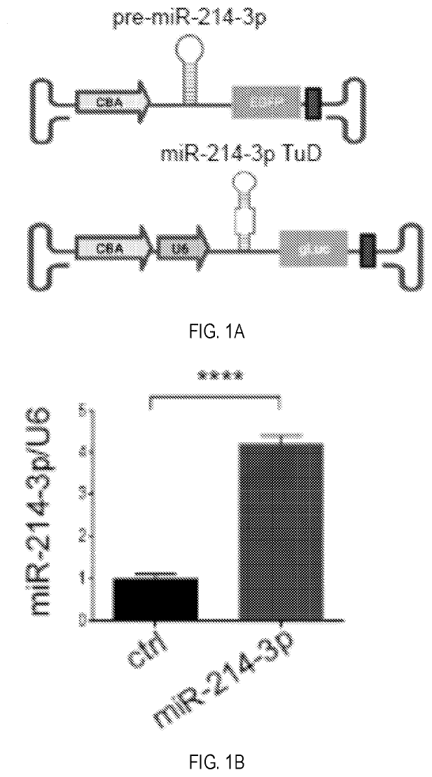

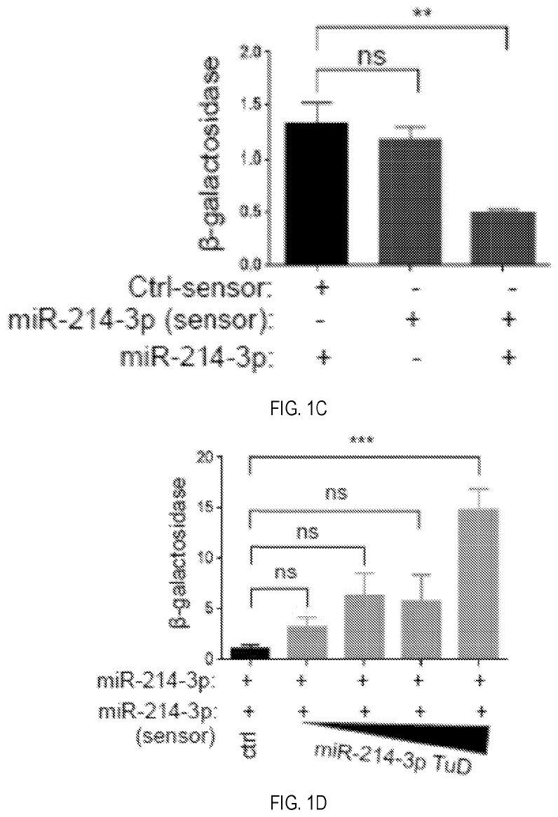

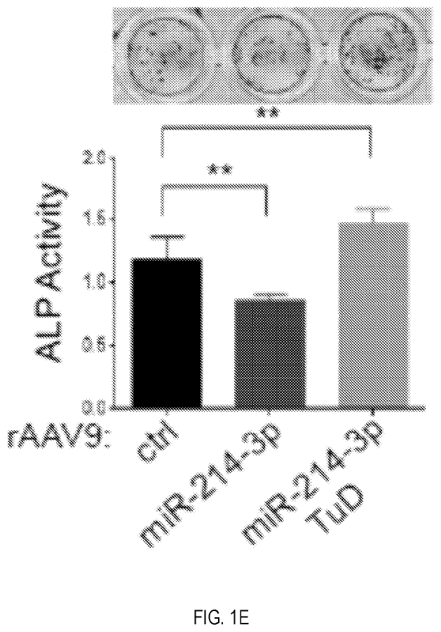

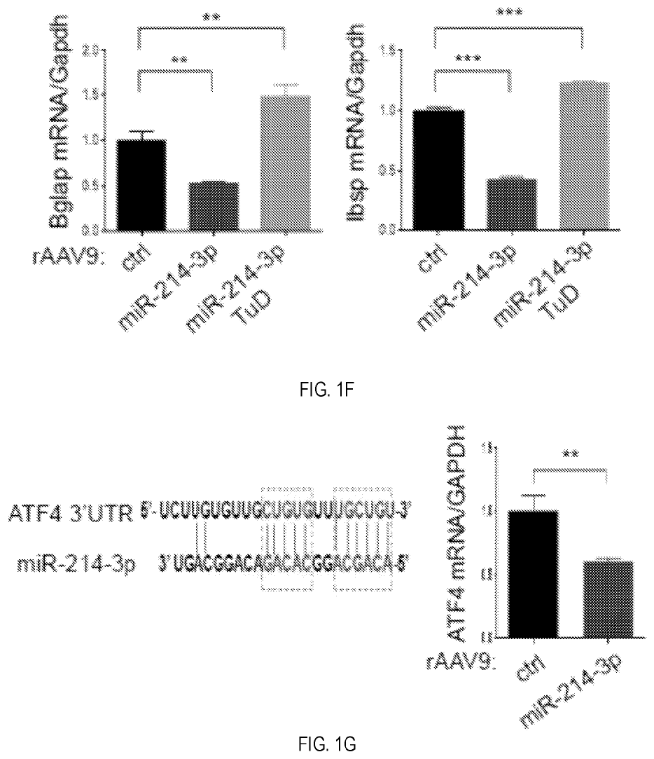

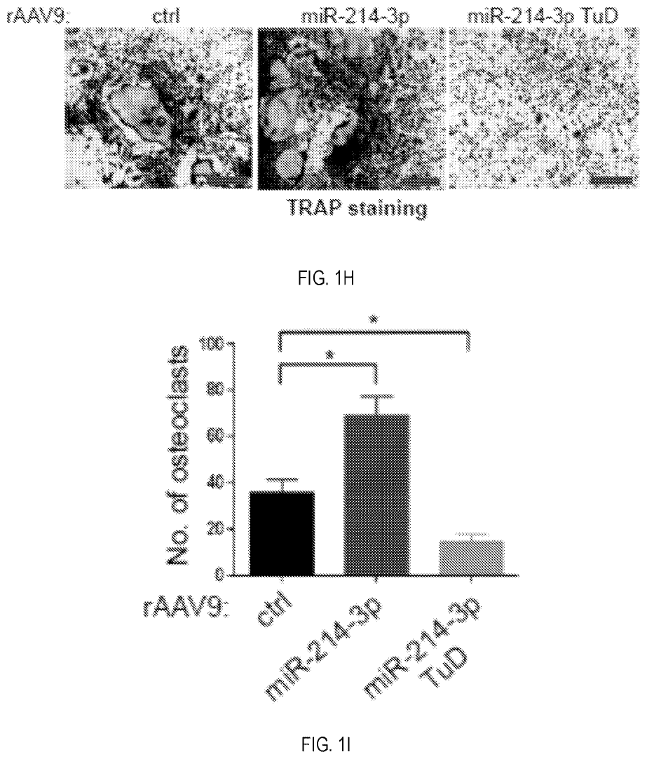

miR-214-3p has been reported to function in both osteoblasts and osteoclasts by inhibiting osteogenesis and promoting osteoclastogenesis. Together with elevated levels of miR-214-3p in osteoporotic bones (FIG. 7A), these data implicate miR-214-3p as a causative factor of osteoporosis. An rAAV-mediated gene transfer platform that enabled long-term expression of pre-miR-214-3p or inhibition of endogenous miR-214-3p by miRNA tough decoys (TuDs) containing multiple tandem miR-214-3p binding sites were used to investigate the role of the miRNAs in osteoporosis. For miR-214-3p expression, pre-miR-214-3p was inserted between the chicken β-actin (CBA) promoter and the EGFP reporter gene; to express TuDs, three tandem miR-214-3p TuDs were inserted between the U6 promoter and the Guassia luciferase (gLuc) reporter gene to track AAV transduced cells or tissues (FIG. 1A). The AAV vector genome expressing miR-214-3p was validated in HEK293 cells using RT-PCR (FIG. 1B). Overexpression of miR-214-3p markedly reduced β-galactosidase activity of the sensor plasmid that contains miR-214-3p120 target sequences in the 3′-UTR of the LacZ reporter gene but did not affect the activity of the control sensor plasmid (FIG. 1C). Moreover, the repression of β-galactosidase activity by miR-214-3p was relieved in a dose-dependent manner by adding an AAV vector expressing miR-214-3p TuD (FIG. 1D). These results demonstrated AAV vector genomes can functionally express miR-214-3p or miR-214-3p TuD.

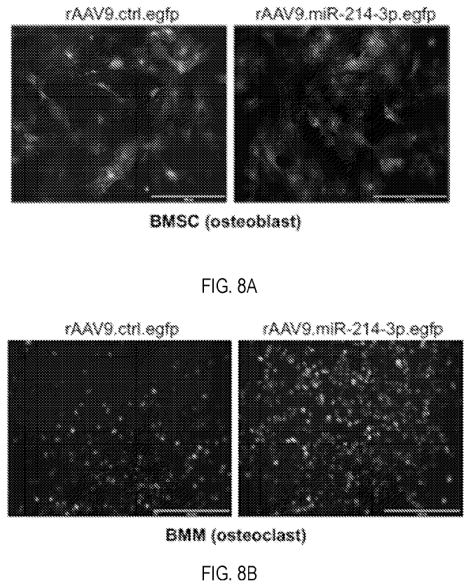

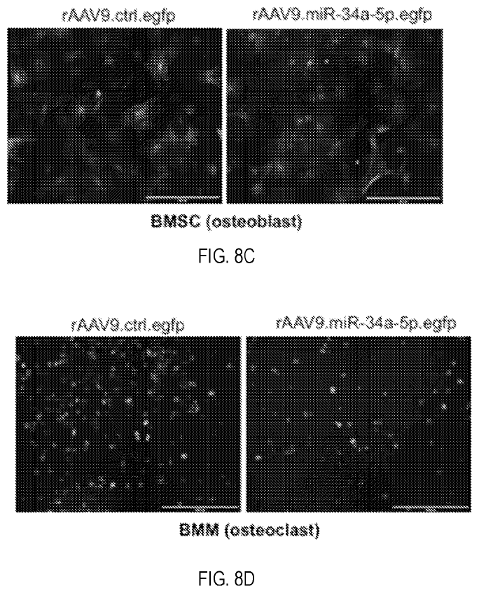

Since the rAAV9 serotype is effective for in vitro and in vivo transduction of osteoblasts and osteoclasts, the test cassettes were then packaged into AAV9 capsids. The in vitro transduction efficiency of rAAV9 vector carrying control or miR-214-3p in mouse bone marrow-derived stromal cells (BMSCs) for osteoblast differentiation and bone marrow monocytes (BMMs) for osteoclast differentiation was validated using EGFP expression (FIGS. 8A-8B). AAV-mediated overexpression of miR-214-3p in BMSCs decreased alkaline phosphatase (ALP) activity and osteogenic gene expression, including Bglap and Ibsp, whereas ALP activity and gene expression were upregulated by miR-214-3p TuD (FIGS. 1E-1F). These results demonstrated that the expression of miR-214-3p or miR-214-3p TuD by the rAAV9 vector effectively regulated osteoblast differentiation in vitro. As miR-214-3p has been reported to target the master transcription factor of osteogenesis ATF, mRNA levels of Atf4 in miR-214-3p-expressing BMSCs were markedly reduced (FIG. 1G), indicating that miR-214-3p inhibits osteoblast differentiation via downregulation of ATF4 expression. Conversely, AAV-mediated expression of miR-214-3p in BMMs increased the number of tartrate-resistant acid phosphatase (TRAP)-positive multinucleated osteoclasts (FIGS. 1H-11), as well as osteoclast gene expression, including Rank and Acp5 (FIG. 1J). This effect was reversed by the expression of miR-214-3p TuD. Since miR-214-3p targets the 3′ UTR of Pten, an inhibitor of RANKL142 activated Akt survival signaling, mRNA levels of Pten were markedly reduced in miR-214-3p143 expressing BMMs (FIG. 1K), indicating that reduced expression of Pten by miR-214-3p promotes osteoclast differentiation due to augmented RANK signaling. Thus, the rAAV9 vector is effective at overexpressing or inhibiting miR-214-3p in both osteoblast and osteoclast progenitors to control osteogenesis and osteoclastogenesis.

rAAV9-Mediated Modulation of miR-34a-5p Regulates Osteoblast and Osteoclast Differentiation In Vitro

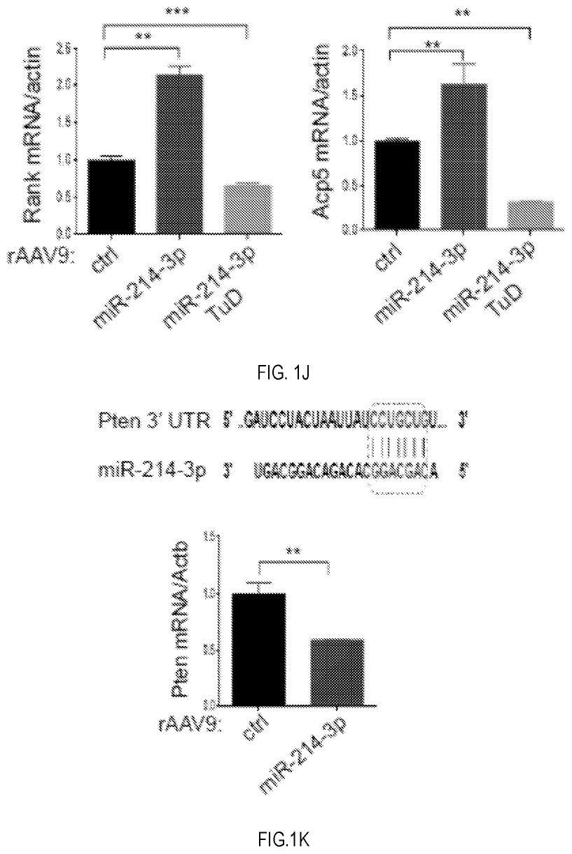

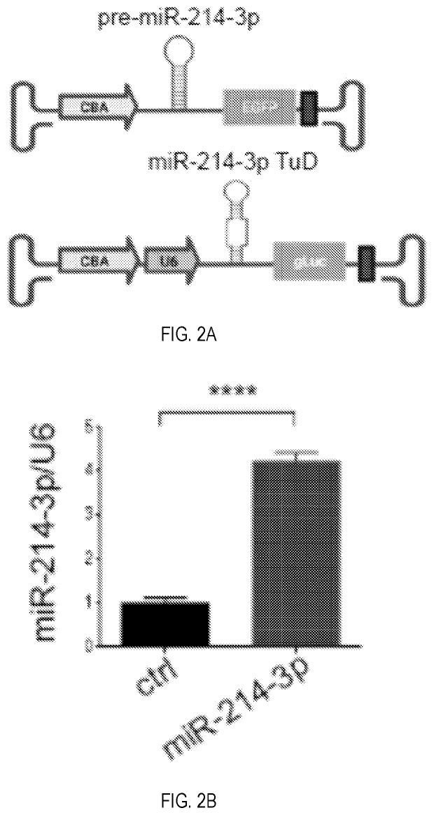

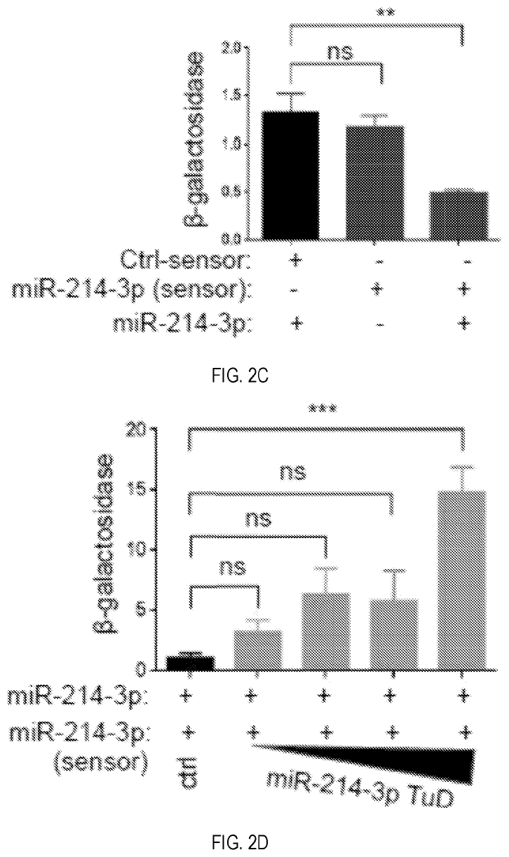

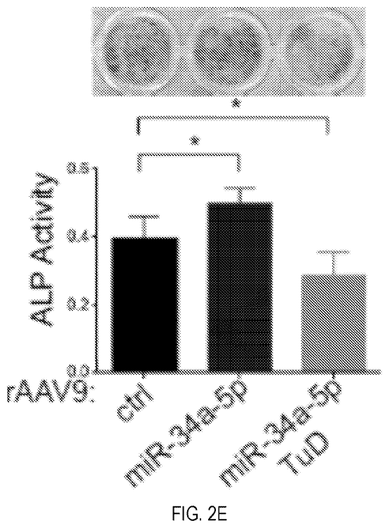

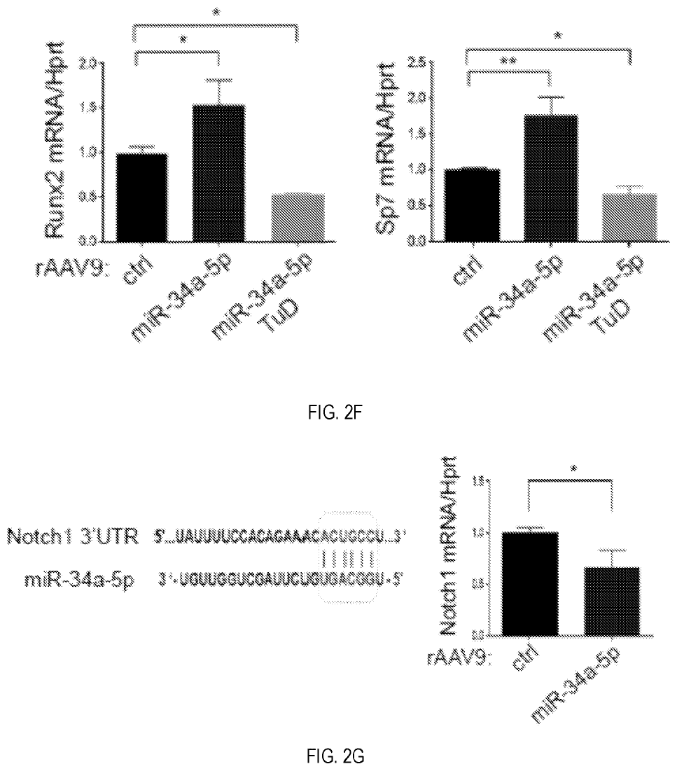

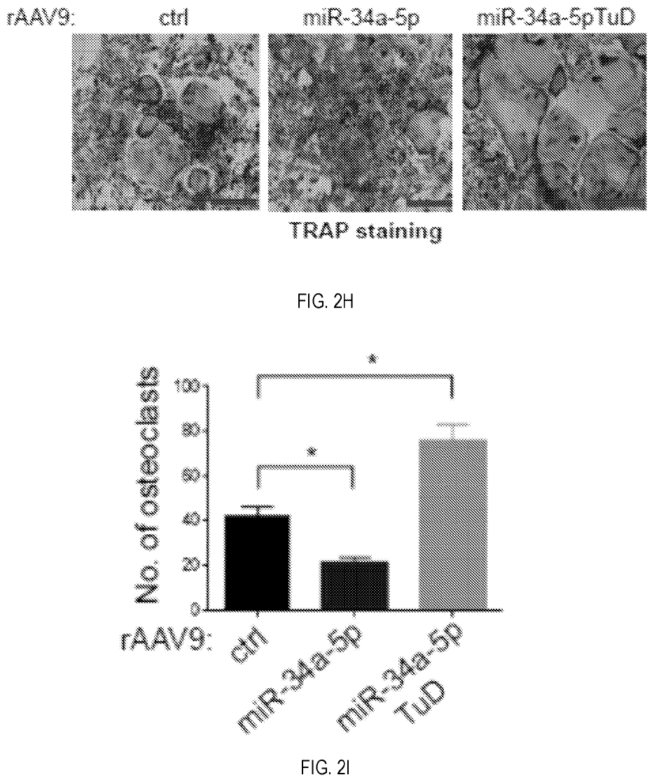

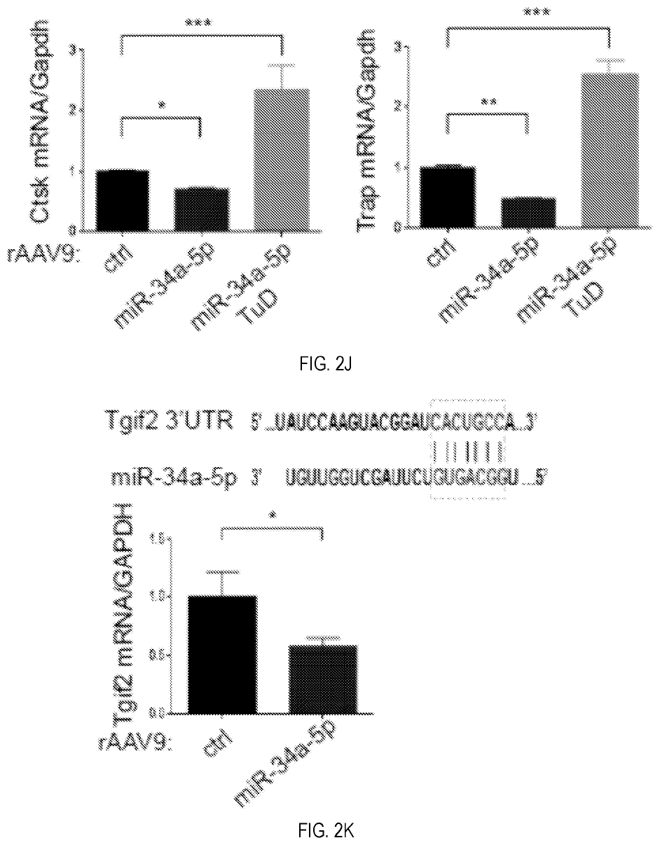

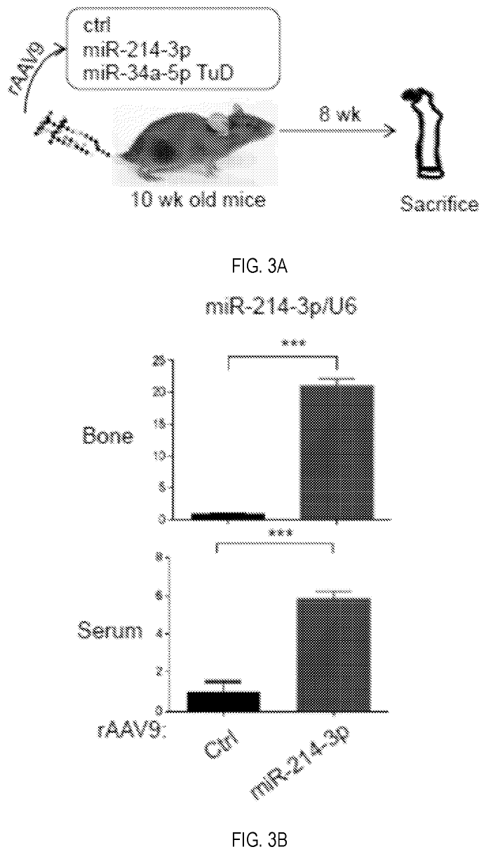

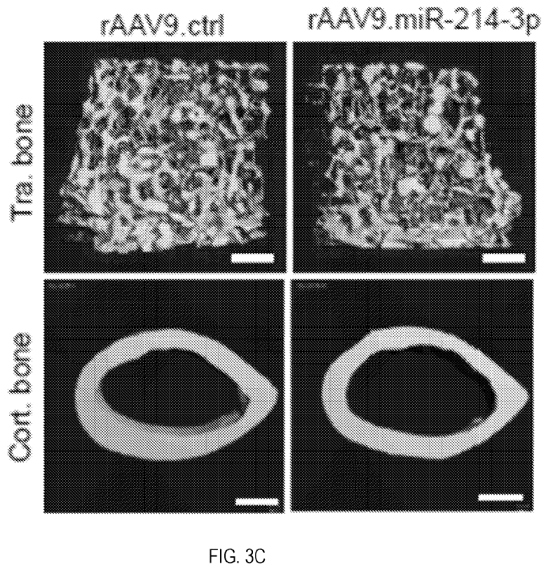

In contrast to miR-214-3p, which decreases osteogenesis and increases osteoclastogenesis, miR-34a-5p increases osteogenesis and decreases osteoclastogenesis and its expression was markedly reduced in osteoporotic bones (FIG. 7B). To test the possibility that miR-34a-5p inhibits osteoporosis, an AAV vector genome containing the expression cassette of miR-34a-5p and the EGFP reporter gene or miR-34a-5p TuD and gLuc reporter gene was constructed (FIG. 2A). The expression and functional activity of these plasmids in HEK293 cells were validated by RT-PCR (FIG. 2B) and by measuring β-galactosidase activity of the sensor plasmid containing miR-34a-5p-target sequences, respectively. Overexpression of miR-34a-5p markedly reduced β-galactosidase activity (FIG. 2C), which was relieved in a dose-dependent manner by expression of miR-34a-5p TuD (FIG. 2D). The test cassettes were then packaged into AAV9 capsids, and their transduction efficiency in osteoblasts (BMSCs) and osteoclasts (BMMs) was validated using EGFP expression in vitro (FIG. 8C-8D). rAAV9-mediated expression of miR-34a-5p increased osteoblast differentiation, as shown by increased ALP activity and osteogenic gene expression, while osteoblast differentiation was markedly decreased by expression of miR-34a-5p TuD (FIG. 2E-2F), demonstrating a positive role of miR-34a-5p in osteogenesis. Since miR-34a-5p targets Notch1 in pancreatic cancers, mRNA levels of Notch1 were markedly reduced in miR-34a-5p-expressing BMSCs (FIG. 2G), indicating that miR-34a-5p promotes osteoblast differentiation via downregulation of Notch1 expression. Conversely, rAAV9-mediated expression of miR-34a-5p decreased osteoclast differentiation, as shown by a decrease in the number of TRAP-positive multinucleated osteoclasts (FIGS. 2H-21) and osteoclast gene expression (FIG. 2J), whereas miR-34a-5p TuD promoted osteoclast differentiation. Since miR-34a-5p targets Tgif2, a key regulator of osteoclast function and differentiation, miR-34a-5p-expressing BMMs showed reduced mRNA levels of Tgif2 (FIG. 2K), indicating that miR-34a-5p inhibits osteoclast differentiation via downregulation of Tgif2 expression (FIG. 2K). Thus, rAAV9-mediated expression of miR-34a-5p or miR-34a-5p TuD is effective in controlling both osteoblast and osteoclast differentiation in vitro.

rAAV9-Mediated Expression of miR-214-3p or miR-34a-5p TuD Induces Bone Loss in Mice

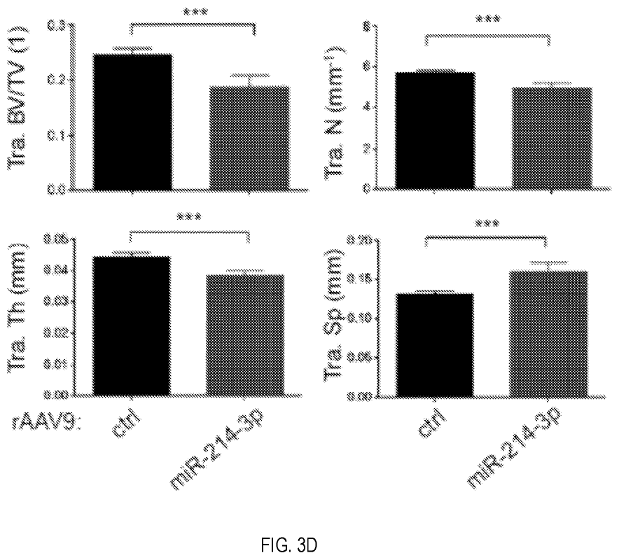

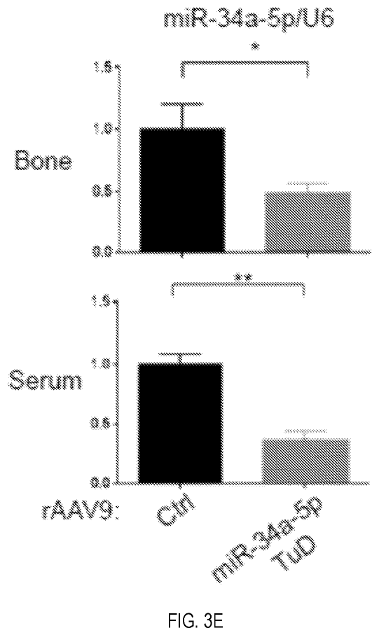

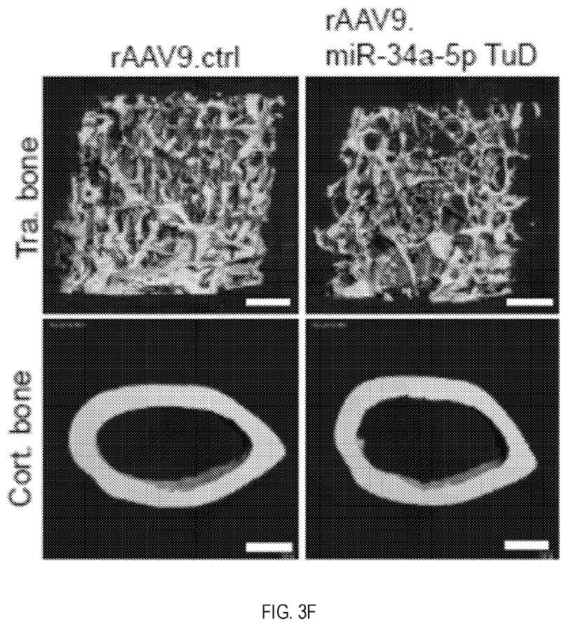

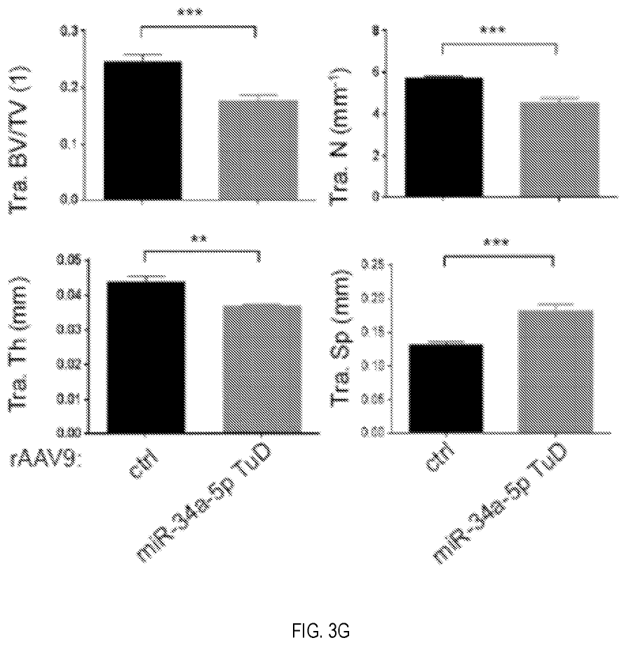

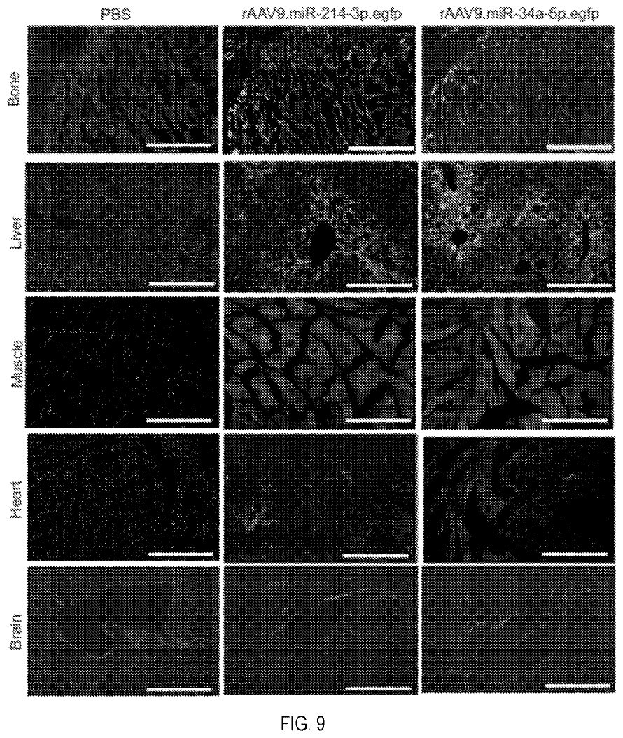

To investigate whether overexpression of miR-214-3p or inhibition of endogenous miR-34a-5p in healthy mice recapitulates bone loss in osteoporotic mice, a single dose of rAAV9 vector carrying control, miR-214-3p, or miR-34a-5p TuD was intravenously (i.v.) injected into 10-week-old mice (FIG. 3A). Eight weeks later, expression levels of miR-214-3p and miR-34a-5p in the tibia and serum (FIGS. 3B and 3E) and femoral bone mass were assessed by RT-PCR and microCT, respectively (FIGS. 3C, 3D, 3F, and 3G). rAAV9's distribution in individual tissues was examined by EGFP expression using fluorescence microscopy, demonstrating AAV's transduction in the bone, heart, liver, and muscle, but not in the brain (FIG. 9). EGFP-expressing cells were observed in the epiphyseal area of the femur, indicating that i.v. injected rAAV9 vector targets osteoblasts and osteoclasts residing in the bone with high bone remodeling activity (FIG. 9). MicroCT analysis revealed low bone mass in the femurs treated with miR-214-3p or miR-34a-5p TuD relative to control, as evidenced by a significant decrease in trabecular bone volume/tissue volume (BV/TV), trabecular numbers, and trabecular thickness (FIGS. 3C, 3D, 3F, and 3G). These results indicate that systemic delivery of the rAAV9 vector effectively expresses miR-214-3p and miR-34a-5p TuD in osteoblast-and osteoclast-lineage cells and results in bone loss in vivo.

rAAV9-Mediated Expression of miR-214-3p TuD or miR-34a-5p Counteracts Bone Loss in Postmenopausal Osteoporosis

Osteoporosis results in severe bone loss and deterioration of bone architecture, increasing the risk of bone fractures. Bone loss in postmenopausal women mainly results from a lack of estrogen, which is normally produced as a part of the menstrual cycle. It acts on osteoclasts as a negative regulator that prevents osteoclast-mediated bone resorption while promoting bone formation due to augmented osteoblast differentiation. Since rAAV9-mediated expression of miR-214-3p TuD or miR-34a-5p resulted in increased osteogenesis and decreased osteoclastogenesis, whether systemic delivery of rAAV9 carrying miR-214-3p TuD or miR-34a-5p can promote osteoblast-mediated bone formation and suppress osteoclast-mediated bone resorption in mice, thereby reversing bone loss in postmenopausal osteoporosis was investigated.

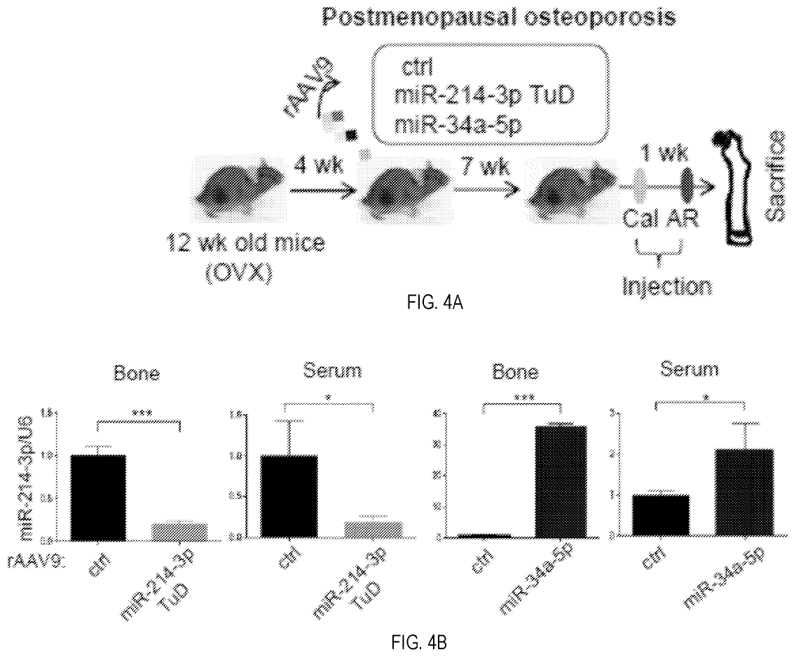

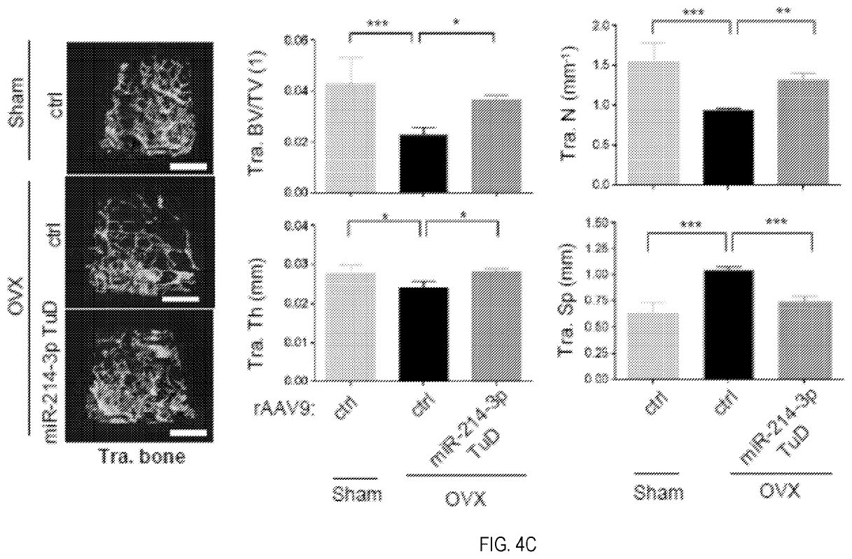

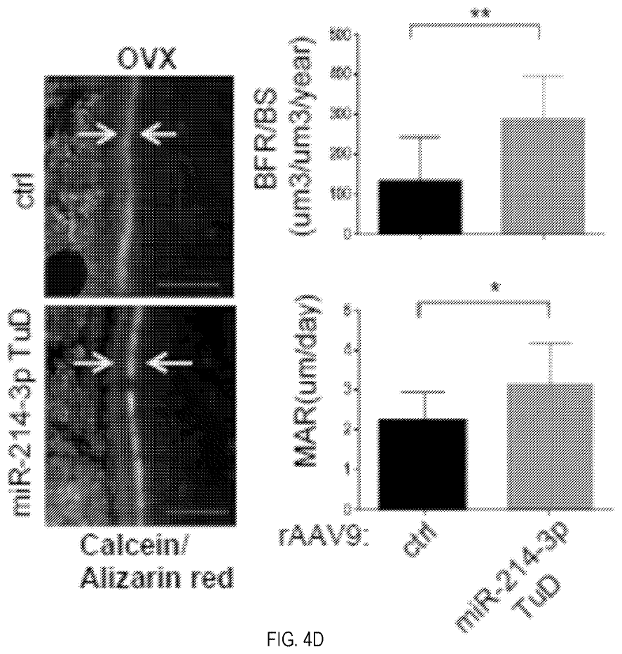

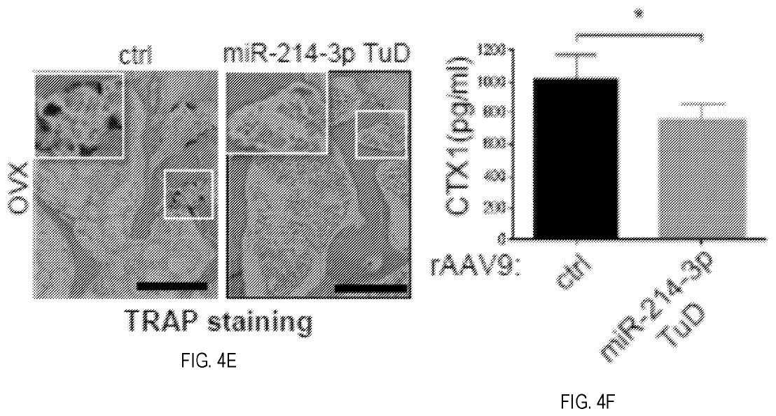

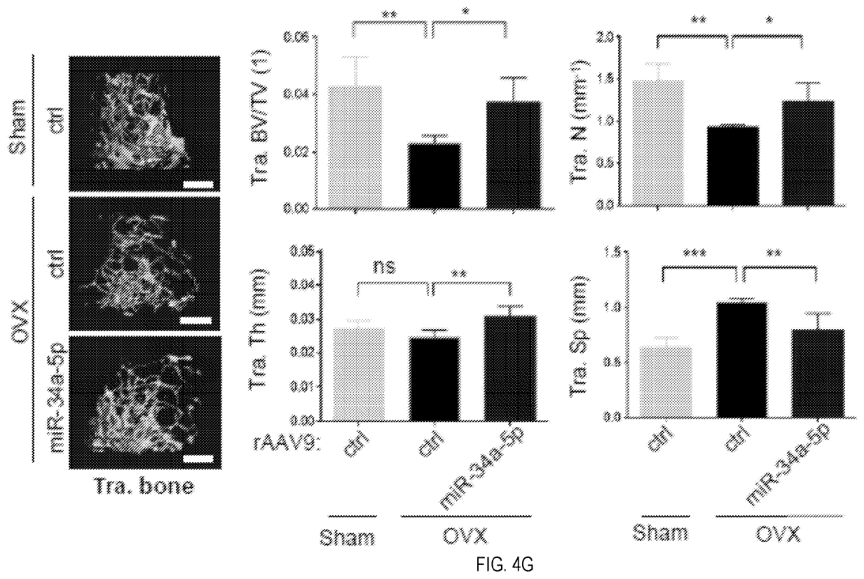

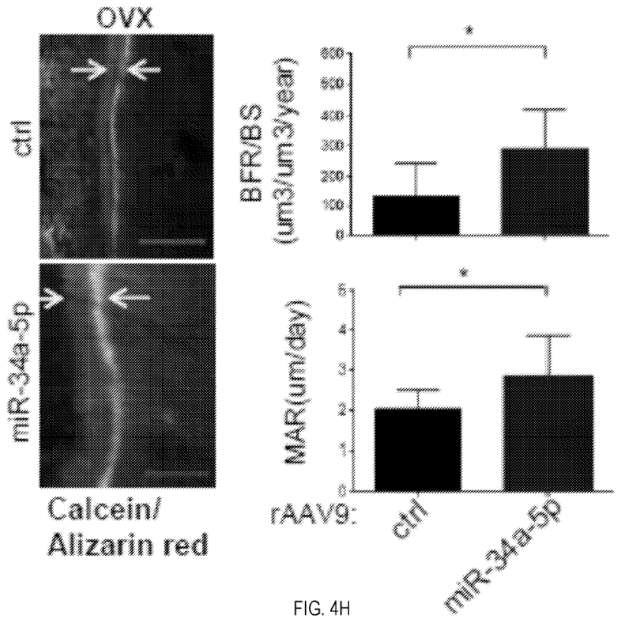

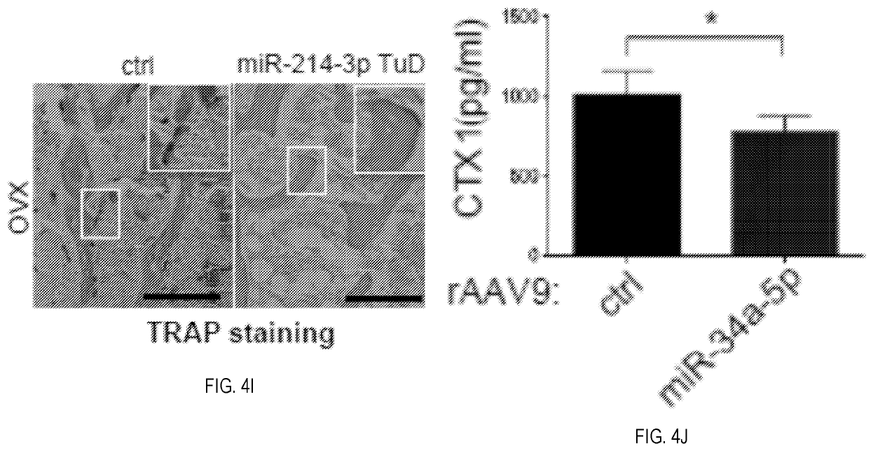

Ovariectomized (OVX) mice are an established model for postmenopausal osteoporosis induced by estrogen deficiency. Sham or OVX surgery was conducted on 12-week-old female mice, and a single dose of rAAV9 carrying control, miR-214-3p TuD, or miR-34a-5p was i.v. injected four weeks post-surgery (FIG. 4A). Eight weeks after injection, a reduced expression of endogenous miR-214-3p or increased expression of miR-34a-5p in the tibia was validated by RT-PCR (FIG. 4B). While control-treated OVX mice showed a significant decrease in trabecular bone mass relative to sham mice, bone loss was almost completely reversed in the femur of OVX mice treated with miR-214-3p TuD or miR-34a-5p, as shown by increased trabecular BV/TV, thickness, and numbers (FIGS. 4C and 4G). Likewise, bone formation rate (BFR) and mineral apposition rate (MAR) were markedly increased in these mice, demonstrating augmented osteoblast activity in vivo (FIGS. 4D and 4H). OVX mice treated with miR-214 TuD or miR-34a-5p also displayed a significant decrease in numbers of TRAP-positive osteoclasts and serum levels of C-terminal telopeptide type I collagen (CTX), demonstrating reduced osteoclast differentiation and resorption activity in vivo, respectively (FIGS. 4E, 4F, 4I, 4J). These results indicate that systemic delivery of rAAV9 carrying miR-214-3p TuD or miR-34a-5p simultaneously promotes osteoblast-mediated bone formation and suppresses osteoclast mediated bone resorption, thereby effectively counteracting bone loss after the onset of postmenopausal osteoporosis.

rAAV9-Mediated Expression of miR-214-3p TuD or miR-34a-5p Reverses Aging-Associated Osteoporosis

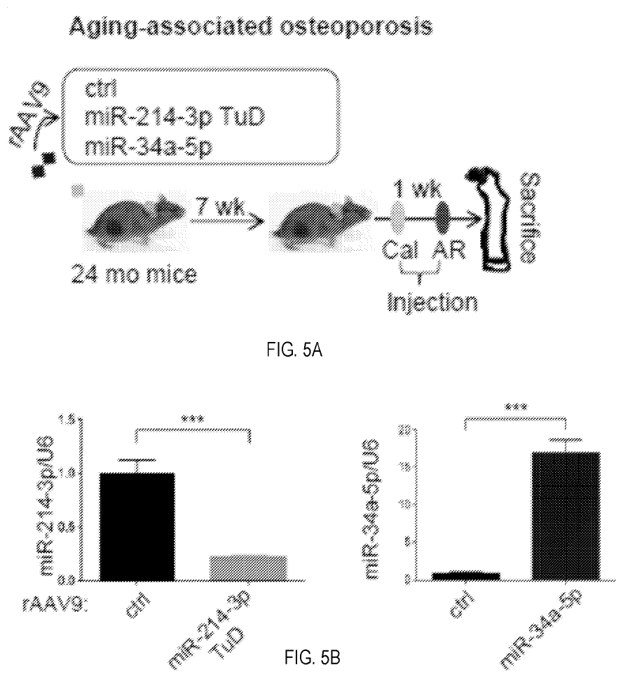

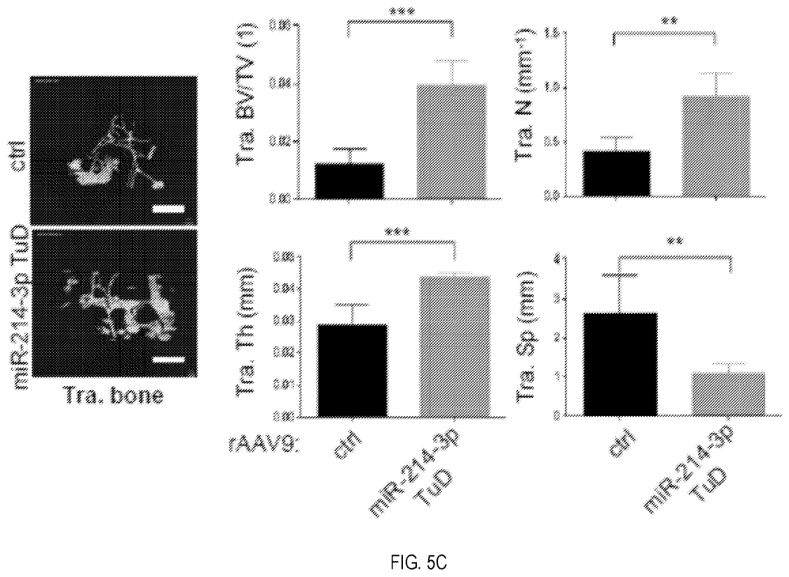

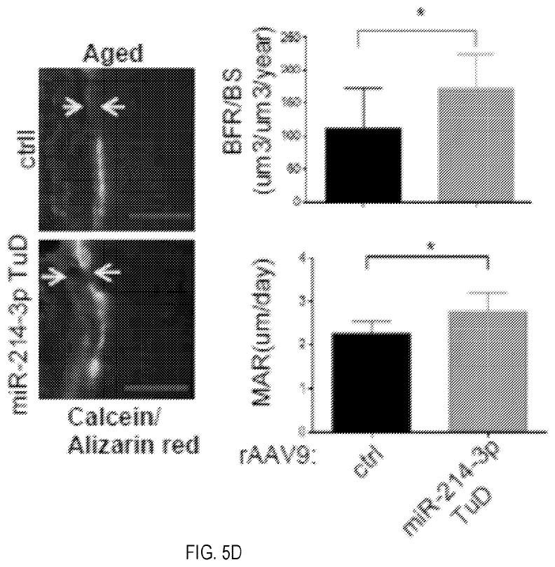

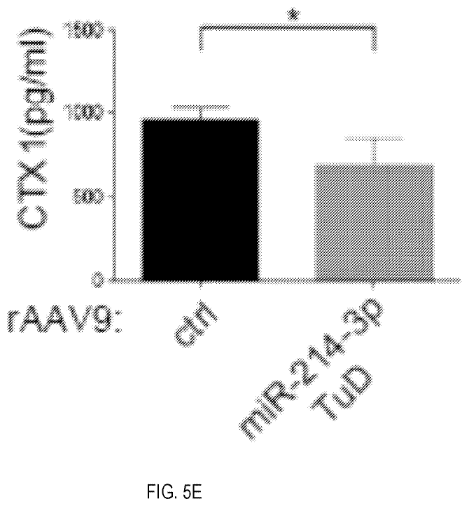

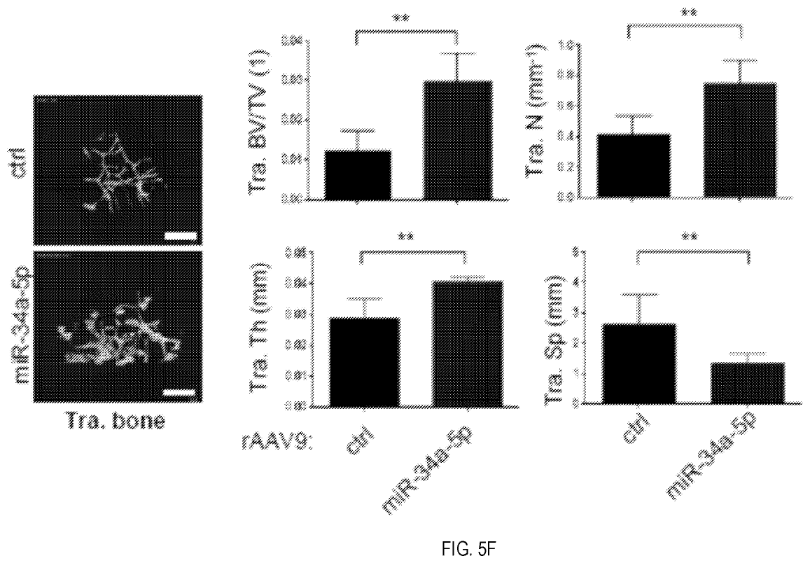

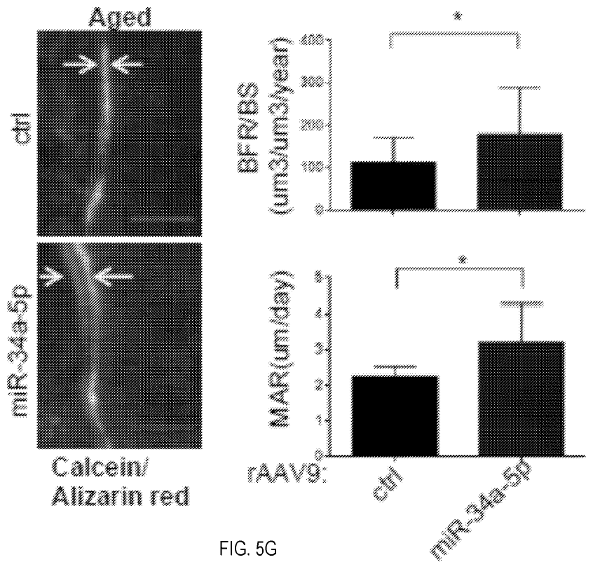

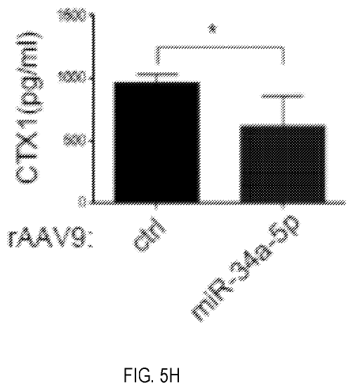

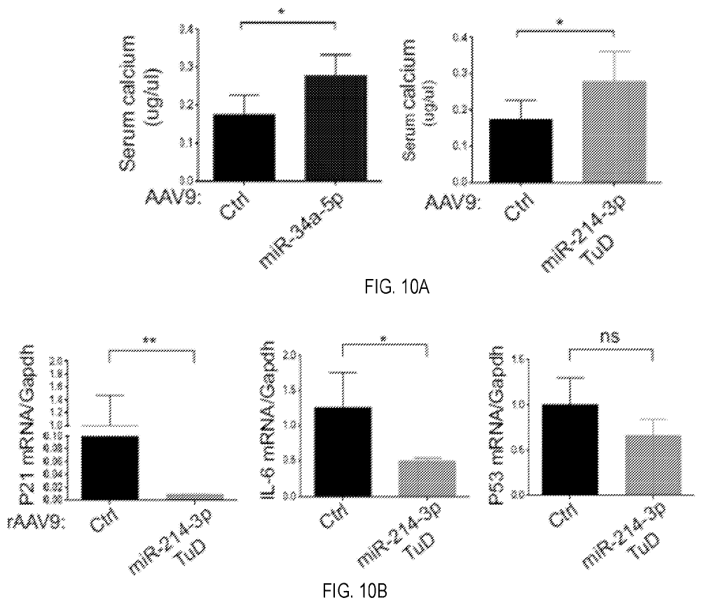

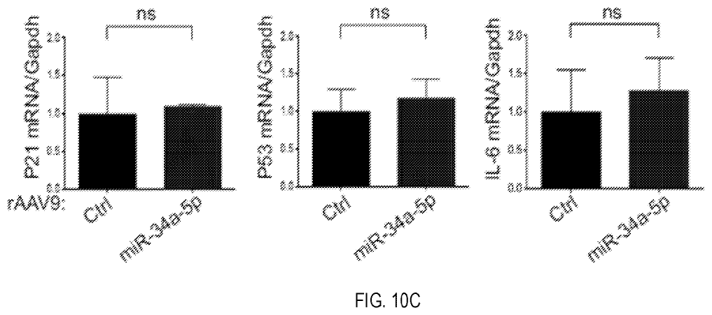

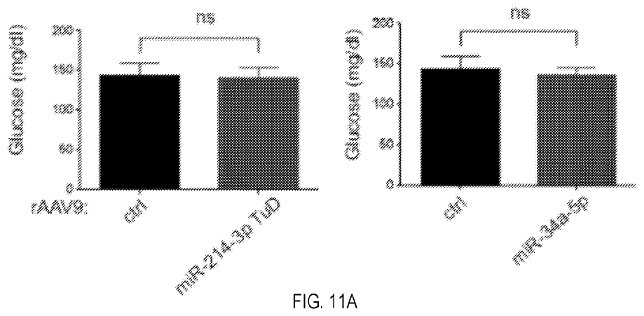

Aging-associated osteoporosis typically occurs after the age of 70 for both men and women mainly due to senescence of skeletal stem cells and progenitors and deficiency of calcium and vitamin D, resulting in decreased osteoblast activity, increased adipogenesis, and impaired DNA repair. To investigate the therapeutic effects of rAAV9 carrying miR-214-3p TuD or miR-34a-5p in a mouse model for senile osteoporosis, 24-month-old male mice were i.v. injected with a single dose of rAAV9 carrying control, miR-214-3p TuD, or miR-34a-5p (FIG. 5A). Eight weeks later, knockdown of endogenous miR-214-3p or overexpression of miR-34a-5p in the tibia was validated by RT-PCR (FIG. 5B). Compared to control-treated mice, miR-214-3p TuD- or miR-34a-5p-treated mice showed a significant increase in trabecular bone mass within the femur, as indicated by increased trabecular BV/TV, thickness, and number (FIGS. 5C and 5F). While femoral BFR and MAR were substantially increased (FIGS. 5D and 5G), these mice displayed reduced levels of serum CTX (FIGS. 5E and 5H). This is accompanied with elevated levels of calcium in the serum (FIG. 10A). These results demonstrated that systemic delivery of rAAV9 carrying miR-214-3p TuD or miR-34a-5p is also effective 242 in reversing aging-associated osteoporosis by promoting osteoblast-mediated bone formation and suppressing osteoclast mediated bone resorption simultaneously. Notably, treatment with miR-214-3p TuD, but not miR-34a-5p, markedly reduced expression of cell senescence genes, including p21 and IL-6, in the tibia (FIGS. 10B-10C), suggesting therapeutic potentials of miR-214-3p TuD to reverse both cell senescence and bone loss in aging-associated osteoporosis. Taken together, inhibition of endogenous miR-214-3p or overexpression of miR-34a-5p via rAAV9-mediated delivery to the bone, which simultaneously promotes bone formation and inhibits bone resorption, is a promising therapeutic approach for the treatment of both postmenopausal and aging-associated osteoporosis.

No Adverse Effects of rAAV9 Carrying miR-214-3p TuD or miR-34a-5p in Mice

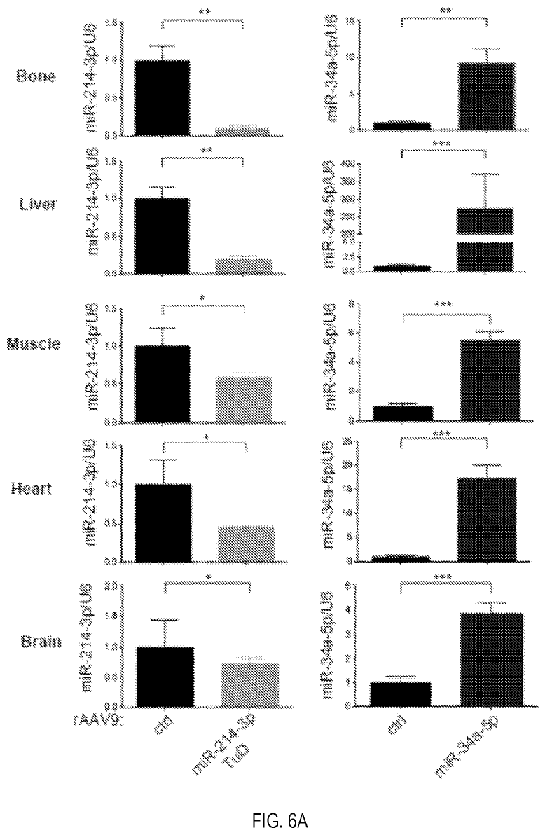

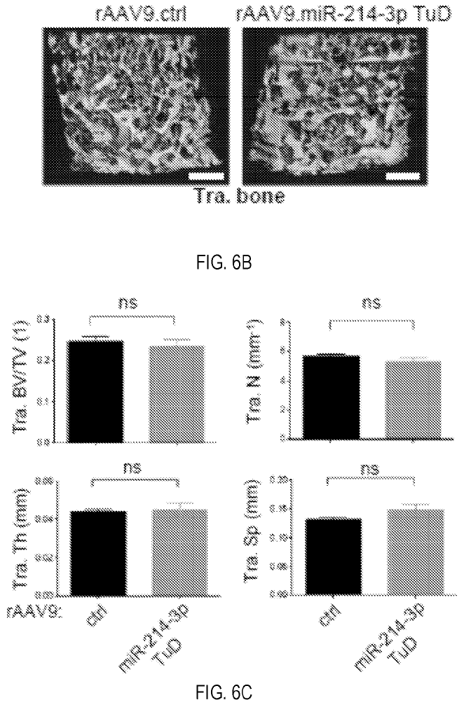

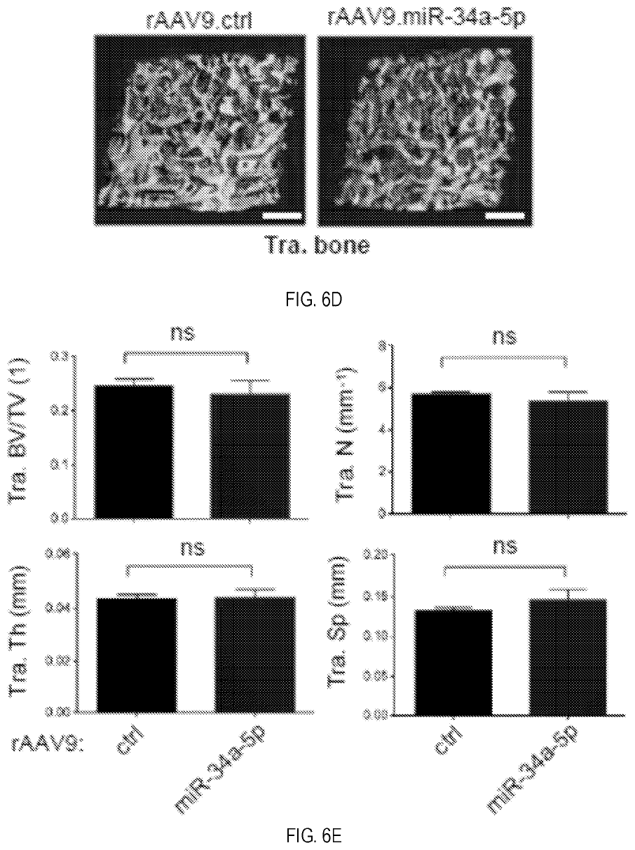

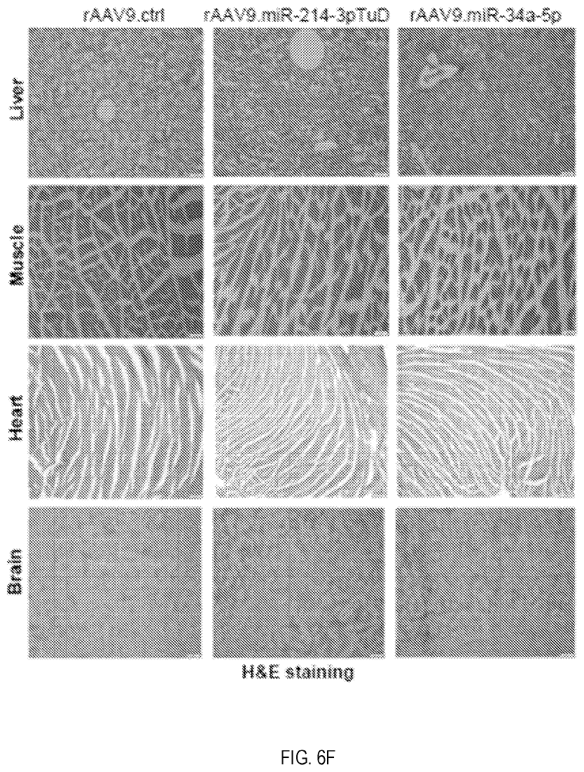

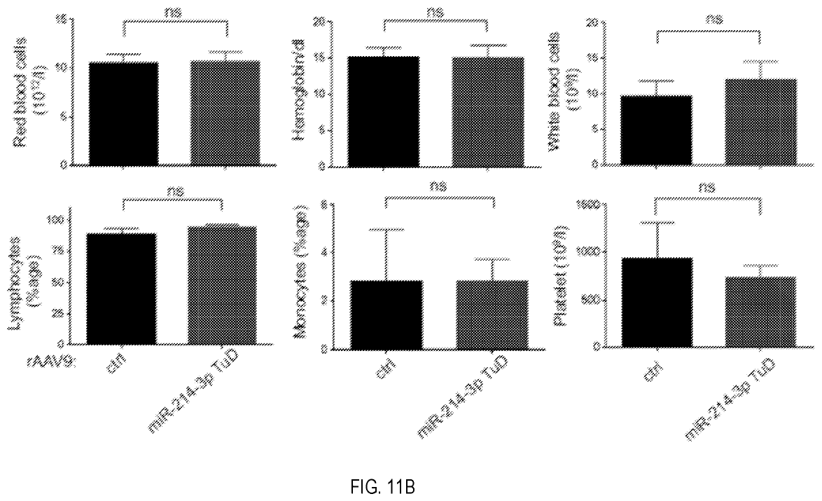

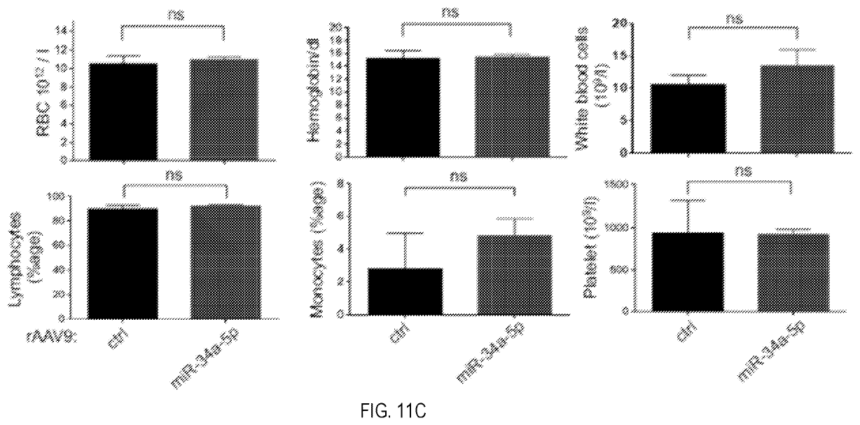

Previous studies have demonstrated that miR-214-3p plays roles in various biological processes, including skeletal development and homeostasis, cancer development, immune responses, skeletal muscle development, ischemic injury in the heart, and angiogenesis, while miR-34a-5p is important for the regulation of multiple target genes involved in cancer cell growth, proliferation, apoptosis and invasion. Given their expression and pleiotropic roles in various tissues, whether rAAV9-mediated inhibition of miR-214-3p or overexpression of miR-34a-5p causes any untoward adverse effects on non-skeletal tissues was investigated. A single dose of rAAV9 vectors carrying control, miR-214-3p TuD, or miR-34a-5p was i.v. injected into 2-month-old healthy mice and eight weeks later, expression levels of miR-214-3p and miR-34a-5p in rAAV9-transduced tissues, including bone, liver, skeletal muscle, heart, and brain, were validated by RT PCR (FIG. 6A). It was observed that i.v. injection of rAAV9 vectors shows the highest transduction efficiency in the liver and the lowest transduction efficiency in the brain. Of note, trabecular BV/TV, thickness, and numbers were all comparable between the femurs treated with control, miR-214-3p TuD, or miR-34a-5p (FIGS. 6B-6E), indicating that the effects of miR-214-3p TuD or miR-34a-5p on normal bone remodeling are minimal in healthy mice. Likewise, these mice did not show any histological abnormalities in rAAV9-transduced non-skeletal tissues (FIG. 6F). Finally, glucose and hemoglobulin levels and numbers of red blood cells, white blood cells, lymphocytes, monocytes, and platelets in the blood were all normal in AAV-treated mice (FIGS. 11A-11C). These results indicate that rAAV9-mediated inhibition of miR-214-3p or overexpression of miR-34a-5p are effective for bone accrual under pathological conditions and do not affect normal bone homeostasis and non275 skeletal tissues.

This example describes that, as miR-214-3p is upregulated and miR-34a-5p is downregulated under osteoporotic conditions, rAAV9-mediated inhibition of endogenous miR-214-3p and overexpression of miR-34a-5p in osteoblasts and osteoclasts were both effective in counteracting bone loss in mouse models for postmenopausal and senile osteoporosis. Of note, tissue morphology and structure of non-skeletal tissues in AAV-treated mice are largely normal, suggesting little to no obvious anatomic off-target side effects of rAAV9 carrying miR-214-3p TuD or miR-34a-5p. Unlike current therapeutic agents for osteoporosis showing numerous potential side effects and limited therapeutic effectiveness due to counterbalanced coupling events between osteoblasts and osteoclasts, inhibition of miR-214-3p or overexpression of miR-34a-5p via a single systemic administration of rAAV9 vector not only maximizes bone accrual capacity in osteoporosis by controlling osteoblast and osteoclast differentiation simultaneously but also minimizes untoward adverse effects in non-skeletal tissues. Moreover, co-injection with rAAV9.miR-214-3p TuD and rAAV9.miR-34a-5p may further increase therapeutic effectiveness in osteoporosis as a combination therapy.

Mechanistically, inhibition of endogenous miR-214-3p upregulates the expression of ATF4 in osteoblasts and PTEN in osteoclasts. On the other hand, overexpression of miR-34a-5p downregulates the expression of Notch1 in osteoblasts and Tgif2 in osteoclasts. miR-214-3p and miR-34a-5p are also involved in the regulation of T cell function and tumorigenesis. miR-214-3p plays a positive role in T cell proliferation and function by downregulating the expression of Pten, an inhibitor of the PI3K-AKT pathway. Conversely, miR-34a-5p functions as a negative regulator of T cell function by downregulating the expression of the genes associated with the NF-κB signalosome. Since activation of T cells under osteoporotic conditions enhances osteoclast-mediated bone resorption, suppression of T cell function by AAV-mediated miR-214-3p inhibition or miR-34a-5p overexpression may contribute to reverse bone loss in osteoporosis. Moreover, miR-214-3p and miR-34a-5p have been reported to regulate the progression of various cancers. miR-214-3p acts as an oncogenic factor in gastric, ovarian, and breast cancers that upregulates PI3K/Akt signaling by suppressing Pten expression, while miR-34a-5p is a tumor suppressor of various cancers, including prostate, esophageal, gastric, breast cancers, which inhibits the expression of CD44, FNDC3B, and IGF2BP3. Thus, the potential of AAV-mediated gene therapy modulating miR-214-3p or miR-34a-5p may extend beyond osteoporosis to various cancers.

TABLE 1
Primers and G-block Sequences

| Oligonucleotides | Sequence (5′to3′) | SEQ ID NO |
|---|---|---|
| TuD34a-5p F | CATCAACACAACCAGCTAAGTCGGACACTGCCACAAG | 15 |
| | TATTCTGGTCACAGAATACAACACAACCAGCTAAGTCG | |
| | GACACTGCCACAAG | |
| TuD34a-5p R | TCATCTTGTGGCAGTGTCCGACTTAGCTGGTTGTGTTG | 16 |
| | TATTCTGTGACCAGAATACTTGTGGCAGTGTCCGACTT | |
| | AGCTGGTTGTGTT | |
| TuDmiR-214-3P F | CATCAACACTGCCTGTCTGGTCGTGCCTGCTGTCAAGT | 17 |
| | ATTCTGGTCACAGAATACAACACTGCCTGTCTGGTCGT | |
| | GCCTGCTGTCAAG | |
| TuDmiR-214-3P R | TCATCTTGACAGCAGGCACGACCAGACAGGCAGTGTT | 18 |
| | GTATTCTGTGACCAGAATACTTGACAGCAGGCACGAC | |
| | CAGACAGGCAGTGTT | |
| 3xmiR-34a-5pT | TCGAGACAACCAGCTAAGACACTGCCAACAACCAGCT | 19 |
| sense | AAGACACTGCCAACAACCAGCTAAGACACTGCCAGC | |
| 3xmiR-34a-5pT | GGCCGCTGGCAGTGTCTTAGCTGGTTGTTGGCAGTGT | 20 |
| antisense | CTTAGCTGGTTGTTGGCAGTGTCTTAGCTGGTTGTC | |
| 3xmiR-214-3p | TCGAGACTGCCTGTCTGTGCCTGCTGTACTGCCTGTCT | 21 |
| sense | GTGCCTGCTGTACTGCCTGTCTGTGCCTGCTGTGC | |
| 3xmiR-214-3p | GGCCGCACAGCAGGCACAGACAGGCAGTACAGCAGG | 22 |
| antisense | CACAGACAGGCAGTACAGCAGGCACAGACAGGCAGTC | |
| miR-34a gBlock | GTCTTTTATTTCAGGTCCCAGATCTTGGCTTCCAAGTG | 23 |
| | CTGGAGGAGTGTGTCATACCTCGGTAGGGTCCACTAC | |
| | ACATCTTTCTCCCGCAGCCTCTCCATCTTCCTGTGACT | |
| | GCGGGCGCCTCAGCCTGGGCTGGCCAGCTGTGAGTA | |
| | ATTCTTTGGCAGTGTCTTAGCTGGTTGTTGTGAGTATT | |
| | AGCTAAGGAAGCAATCAGCAAGTATACTGCCCTAGAA | |
| | GTGCTGCACATTGTTGGGCCGAGAAGGAAAAGGTCAG | |
| | AGGTCAGCAACGCCCACACCCCTGAGAGGCGCTGGA | |
| | CTTGCGGAGCTGCTCGACCATACTGGTGGGTATGGGA | |
| | TGCTGGGGAGGCTGGTGTTACTGCAGGGGATCCGGT | |
| | GGTGGTGC | |
| miR-214 gBlock | GTCTTTTATTTCAGGTCCCAGATCTTTAGTTCCATAATG | 24 |
| | TTTTAATGTTTAATTCTATTGTGTGTTTCTCTCCTTTCCC | |
| | TTTATCCCCCTGTCCTTCCCCCCTAAATCACCAAATCT | |
| | GGAAAACAGGCTGATTGTATCTGTCCCTGAGCAAAGG | |
| | AAACCTGAAGGACCCAAGGGCCTGGCTGGACAGAGTT | |
| | GTCATGTGTCTGCCTGTCTACACTTGCTGTGCAGAACA | |
| | TCCGCTCACCTGTACAGCAGGCACAGACAGGCAGTCA | |
| | CATGACAACCCAGCCTGAATGACCACCAGCCATTGAA | |
| | AGAAAGCTGCCCTCACAACATAGCATCTACACCAAGAG | |
| | CTACAACCACAGTGAGGGGGTTGGGGGGCCTGGGGT | |
| | TTGAAACTGTTGGCTTATTAAAAGAAAAACTCGTATGTA | |
| | ATCCCTGCAGGGGATCCGGTGGTGGTGC | |

REPRESENTATIVE SEQUENCES

>mature miR-34a-5p nucleic acid sequence (SEQ ID NO: 1)
5′-UGGCAGUGUCUUAGCUGGUUGU-3′
>miR-34a-5p encoding nucleic acid sequence (SEQ ID NO: 2)
GGCCAGCUGUGAGUGUUUCUUUGGCAGUGUCUUAGCUGGUUGUUGUGAGCAAUA
GUAAGGAAGCAAUCAGCAAGUAUACUGCCCUAGAAGUGCUGCACGUUGUGGGGC
CC
>3x miR-34a-5p encoding nucleic acid sequence (SEQ ID NO: 3)
tggcttccaagtgctggaggagtgtgtcatacctcggtagggtccactacacatctttctcccgcagcctctccatcttcctgtgactggggc
gcctcagcctgggctggccagctgtgagtaattctttggcagtgtcttagctggttgttgtgagtattagctaaggaagcaatcagcaagtata
ctgccctagaagtgctgcacattgttgggccgagaaggaaaaggtcagaggtcagcaacgcccacacccctgagaggcgctggacttgc
ggagctgctcgaccatactggtgggtatgggatgctggggaggctggtgtta
>3x miR-34a rAAV vector nucleic acid sequence (SEQ ID NO: 4)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtcgacattgattattgactctggtcgttacataa
cttacggtaaatggcccgcctggctgaccgcccaacgaccccgcccattgacgtcaataatgacgtatgttcccatagtaacgccaatagg
gactttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctactcgaggcca
cgttctgcttcactctccccatctcccccccctccccacccccaattttgtatttatttattttttaattattttgtgcagcgatgggggcggggggg
gggggggggggggcgcgcgccaggcggggcggggcggggcgaggggcggggcggggcgaggcggagaggtgcggcggcag
ccaatcagagcggcgcgctccgaaagtttccttttatggcgaggcggcggcggcggcggccctataaaaagcgaagcgcgcggcggg
cgggagcgggatcagccaccgcggtggcggccctagagtcgatcgaggaactgaaaaaccagaaagttaactggtaagtttagtcttttt
gtcttttatttcaggtcccagatcttggcttccaagtgctggaggagtgtgtcatacctcggtagggtccactacacatctttctcccgcagcct
ctccatcttcctgtgactgcgggcgcctcagcctgggctggccagctgtgagtaattctttggcagtgtcttagctggttgttgtgagtattagc
taaggaagcaatcagcaagtatactgccctagaagtgctgcacattgttgggccgagaaggaaaaggtcagaggtcagcaacgcccaca
cccctgagaggcgctggacttgcggagctgctcgaccatactggtgggtatgggatgctggggaggctggtgttactgcaggggatccg
gtggtggtgcaaatcaaagaactgctcctcagtggatgttgcctttacttctaggcctgtacggaagtgttacttctgctctaaaagctgcgga
attgtacccgcggccgatccaccggtcgccaccatggtgagcaagggcgaggagctgttcaccggggggtgcccatcctggtcgagct
ggacggcgacgtaaacggccacaagttcagcgtgtccggcgagggcgagggcgatgccacctacggcaagctgaccctgaagttcatc
tgcaccaccggcaagctgcccgtgccctggcccaccctcgtgaccaccctgacctacggcgtgcagtgcttcagccgctaccccgacca
catgaagcagcacgacttcttcaagtccgccatgcccgaaggctacgtccaggagcgcaccatcttcttcaaggacgacggcaactacaa
gacccgcgccgaggtgaagttcgagggcgacaccctggtgaaccgcatcgagctgaagggcatcgacttcaaggaggacggcaacat
cctggggcacaagctggagtacaactacaacagccacaacgtctatatcatggccgacaagcagaagaacggcatcaaggtgaacttca
agatccgccacaacatcgaggacggcagcgtgcagctcgccgaccactaccagcagaacacccccatcggcgacggccccgtgctgc
tgcccgacaaccactacctgagcacccagtccgccctgagcaaagaccccaacgagaagcgcgatcacatggtcctgctggagttcgtg
accgccgccgggatcactctcggcatggacgagctgtacaagtaaagcggccatcaagcttatcgataccgtcgactagagctcgctgat
cagcctcgactgtgccttctagttgccagccatctgttgtttgcccctcccccgtgccttccttgaccctggaaggtgccactcccactgtcctt
tcctaataaaatgaggaaattgcatcgcattgtctgagtaggtgtcattctattctggggggtggggtggggcaggacagcaagggggagg
attgggaagacaattaggtagataagtagcatgggggttaatcattaactacaaggaacccctagtgatggagttggccactccctctctgc
gcgctcgctcgctcactgaggccgggcgaccaaaggtcgcccgacgcccgggctttgcccgggcggcctcagtgagcgagcgagcgc
gcag
>mmu-miR-214-3p binding site nucleic acid sequence (SEQ ID NO: 5)
catcaacactgcctgtctggtcgtgcctgctgtcaagtattctggtcacagaatacaacactgcctgtctggtcgtgcctgctgtcaag
>miR-214-3p TuD rAAV vector nucleic acid sequence (SEQ ID NO: 6)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtcgacattgattattgactctggtcgttacataa
cttacggtaaatggcccgcctggctgaccgcccaacgaccccgcccattgacgtcaataatgacgtatgttcccatagtaacgccaatagg
gactttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctactcgaggcca
cgttctgcttcactctccccatctcccccccctccccacccccaattttgtatttatttattttttaattattttgtgcagcgatgggggcggggggg
gggggggggggggcgcgcgccaggcggggcggggcggggcgaggggcggggcggggcgaggcggagaggtgcggcggcag
ccaatcagagcggcgcgctccgaaagtttccttttatggcgaggcggcggcggcggcggccctataaaaagcgaagcgcgcggcggg
cgggagcgggatcagccaccgcggtggcggcctagagtcgacgaggaactgaaaaaccagaaagttaactggtaagtttagtctttttgt
cttttatttcaggtcaattccccagtggaaagacgcgcaggcaaaacgcaccacgtgacggagcgtgaccgcgcgccgagcgcgcgcca
aggtcgggcaggaagagggcctatttcccatgattccttcatatttgcatatacgatacaaggctgttagagagataattagaattaatttgact
gtaaacacaaagatattagtacaaaatacgtgacgtagaaagtaataatttcttgggtagtttgcagttttaaaattatgttttaaaatggactatc
atatgcttaccgtaacttgaaagtatttcgatttcttgggtttatatatcttgtggaaaggacgcgggatccgacggcgctaggatcatcaacac
tgcctgtctggtcgtgcctgctgtcaagtattctggtcacagaatacaacactgcctgtctggtcgtgcctgctgtcaagatgatcctagcgcc
gtcttttttaagcttgatccggtggtggtgcaaatcaaagaactgctcctcagtggatgttgcctttacttctaggcctgtacggaagtgttacttc
tgctctaaaagctgcggaattgtacccgcggccgatccaccggtcgccaccatctagcatgggagtcaaagttctgtttgccctgatctgcat
cgctgtggccgaggccaagcccaccgagaacaacgaagacttcaacatcgtggccgtggccagcaacttcgcgaccacggatctcgat
gctgaccgcgggaagttgcccggcaagaagctgccgctggaggtgctcaaagagatggaagccaatgcccggaaagctggctgcacc
aggggctgtctgatctgcctgtcccacatcaagtgcacgcccaagatgaagaagttcatcccaggacgctgccacacctacgaaggcgac
aaagagtccgcacagggcggcataggcgaggcgatcgtcgacattcctgagattcctgggttcaaggacttggagcccatggagcagttc
atcgcacaggtcgatctgtgtgtggactgcacaactggctgcctcaaagggcttgccaacgtgcagtgttctgacctgctcaagaagtggct
gccgcaacgctgtgcgacctttgccagcaagatccagggccaggtggacaagatcaagggggccggtggtgactagctcgacgctgat
cagcctcgactgtgccttctagttgccagccatctgttgtttgcccctcccccgtgccttccttgaccctggaaggtgccactcccactgtcctt
tcctaataaaatgaggaaattgcatcgcattgtctgagtaggtgtcattctattctggggggtggggtggggcaggacagcaagggggagg
attgggaagacaattaggtagataagtagcatggcgggttaatcattaactacaaggaacccctagtgatggagttggccactccctctctgc
gcgctcgctcgctcactgaggccgggcgaccaaaggtcgcccgacgcccgggctttgcccgggcggcctcagtgagcgagcgagcgc
gcag
>miR-34a-5p TuD rAAV vector nucleic acid sequence (SEQ ID NO: 7)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtcgacattgattattgactctggtcgttacataa
cttacggtaaatggcccgcctggctgaccgcccaacgaccccgcccattgacgtcaataatgacgtatgttcccatagtaacgccaatagg
gactttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctactcgaggcca
cgttctgcttcactctccccatctcccccccctccccacccccaattttgtatttatttattttttaattattttgtgcagcgatgggggcggggggg
gggggggggggggcgcgcgccaggcggggcggggcggggcgaggggcggggcggggcgaggcggagaggtgcggcggcag
ccaatcagagcggcgcgctccgaaagtttccttttatggcgaggcggcggcggcggcggccctataaaaagcgaagcgcgcggcggg
cgggagcgggatcagccaccgcggtggcggcctagagtcgacgaggaactgaaaaaccagaaagttaactggtaagtttagtctttttgt
cttttatttcaggtcaattccccagtggaaagacgcgcaggcaaaacgcaccacgtgacggagcgtgaccgcgcgccgagcgcgcgcca
aggtcgggcaggaagagggcctatttcccatgattccttcatatttgcatatacgatacaaggctgttagagagataattagaattaatttgact
gtaaacacaaagatattagtacaaaatacgtgacgtagaaagtaataatttcttgggtagtttgcagttttaaaattatgttttaaaatggactatc
atatgcttaccgtaacttgaaagtatttcgatttcttgggtttatatatcttgtggaaaggacgcgggatccgacggcgctaggatcatcaacac
aaccagctaagtcggacactgccacaagtattctggtcacagaatacaacacaaccagctaagtcggacactgccacaagatgatcctag
cgccgtcttttttaagcttgatccggtggtggtgcaaatcaaagaactgctcctcagtggatgttgcctttacttctaggcctgtacggaagtgtt
acttctgctctaaaagctgcggaattgtacccgcggccgatccaccggtcgccaccatctagcatgggagtcaaagttctgtttgccctgatc
tgcatcgctgtggccgaggccaagcccaccgagaacaacgaagacttcaacatcgtggccgtggccagcaacttcgcgaccacggatct
cgatgctgaccgcgggaagttgcccggcaagaagctgccgctggaggtgctcaaagagatggaagccaatgcccggaaagctggctg
caccaggggctgtctgatctgcctgtcccacatcaagtgcacgcccaagatgaagaagttcatcccaggacgctgccacacctacgaagg
cgacaaagagtccgcacagggggcataggcgaggcgatcgtcgacattcctgagattcctgggttcaaggacttggagcccatggagc
agttcatcgcacaggtcgatctgtgtgtggactgcacaactggctgcctcaaagggcttgccaacgtgcagtgttctgacctgctcaagaag
tggctgccgcaacgctgtgcgacctttgccagcaagatccagggccaggtggacaagatcaagggggccggtggtgactagctcgacg
ctgatcagcctcgactgtgccttctagttgccagccatctgttgtttgcccctcccccgtgccttccttgaccctggaaggtgccactcccactg
tcctttcctaataaaatgaggaaattgcatcgcattgtctgagtaggtgtcattctattctggggggtggggtggggcaggacagcaagggg
gaggattgggaagacaattaggtagataagtagcatggcgggttaatcattaactacaaggaacccctagtgatggagttggccactccctc
tctgcgcgctcgctcgctcactgaggccgggcgaccaaaggtcgcccgacgcccgggctttgcccgggcggcctcagtgagcgagcg
agcgcgcag
>3x miR-214-3p rAAV nucleic acid sequence (SEQ ID NO: 8)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtcgacattgattattgactctggtcgttacataa
cttacggtaaatggcccgcctggctgaccgcccaacgaccccgcccattgacgtcaataatgacgtatgttcccatagtaacgccaatagg
gactttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctactcgaggcca
cgttctgcttcactctccccatctcccccccctccccacccccaattttgtatttatttattttttaattattttgtgcagcgatgggggcggggggg
gggggggggggggcgcgcgccaggcggggcggggcggggcgaggggcggggcggggcgaggcggagaggtgcggcggcag
ccaatcagagcggcgcgctccgaaagtttccttttatggcgaggcggcggcggcggcggccctataaaaagcgaagcgcgcggggg
cgggagcgggatcagccaccgcggtggcggccctagagtcgatcgaggaactgaaaaaccagaaagttaactggtaagtttagtcttttt
gtcttttatttcaggtcccagatctttagttccataatgttttaatgtttaattctattgtgtgtttctctcctttccctttatccccctgtccttc
ccccctaaatcaccaaatctggaaaacaggctgattgtatctgtccctgagcaaaggaaacctgaaggacccaagggcctggctggacagagttgtc
atgtgtctgcctgtctacacttgctgtgcagaacatccgctcacctgtacagcaggcacagacaggcagtcacatgacaacccagcctgaat
gaccaccagccattgaaagaaagctgccctcacaacatagcatctacaccaagagctacaaccacagtgagggggttggggggcctgg
ggtttgaaactgttggcttattaaaagaaaaactcgtatgtaatccctgcaggggatccggtggtggtgcaaatcaaagaactgctcctcagt
ggatgttgcctttacttctaggcctgtacggaagtgttacttctgctctaaaagctgcggaattgtacccgcggccgatccaccggtcgccac
catggtgagcaagggcgaggagctgttcaccggggtggtgcccatcctggtcgagctggacggcgacgtaaacggccacaagttcagc
gtgtccggcgagggcgagggcgatgccacctacggcaagctgaccctgaagttcatctgcaccaccggcaagctgcccgtgccctggc
ccaccctcgtgaccaccctgacctacggcgtgcagtgcttcagccgctaccccgaccacatgaagcagcacgacttcttcaagtccgccat
gcccgaaggctacgtccaggagcgcaccatcttcttcaaggacgacggcaactacaagacccgcgccgaggtgaagttcgagggcgac
accctggtgaaccgcatcgagctgaagggcatcgacttcaaggaggacggcaacatcctggggcacaagctggagtacaactacaaca
gccacaacgtctatatcatggccgacaagcagaagaacggcatcaaggtgaacttcaagatccgccacaacatcgaggacggcagcgtg
cagctcgccgaccactaccagcagaacacccccatcggcgacggccccgtgctgctgcccgacaaccactacctgagcacccagtccg
ccctgagcaaagaccccaacgagaagcgcgatcacatggtcctgctggagttcgtgaccgccgccgggatcactctcggcatggacga
gctgtacaagtaaagcggccatcaagcttatcgataccgtcgactagagctcgctgatcagcctcgactgtgccttctagttgccagccatct
gttgtttgcccctcccccgtgccttccttgaccctggaaggtgccactcccactgtcctttcctaataaaatgaggaaattgcatcgcattgtct
gagtaggtgtcattctattctggggggtggggtggggcaggacagcaagggggaggattgggaagacaattaggtagataagtagcatg
gcgggttaatcattaactacaaggaacccctagtgatggagttggccactccctctctgcgcgctcgctcgctcactgaggccgggcgacc
aaaggtcgcccgacgcccgggctttgcccgggcggcctcagtgagcgagcgagcgcgcag
>hsa-miR-34a-5p sequence (SEQ ID NO: 9)
UGGCAGUGUCUUAGCUGGUUGU
>hsa-miR-34a pri-miRNA sequence (SEQ ID NO: 10)
GGCCAGCUGUGAGUGUUUCUUUGGCAGUGUCUUAGCUGGUUGUUGUGAGCAAUA
GUAAGGAAGCAAUCAGCAAGUAUACUGCCCUAGAAGUGCUGCACGUUGUGGGGC
CC
>hsa-miR-214-3p mature nucleic acid sequence (SEQ ID NO: 11)
ACAGCAGGCACAGACAGGCAGU
>hsa-miR-214 pri-miRNA sequence (SEQ ID NO: 12)
GGCCUGGCUGGACAGAGUUGUCAUGUGUCUGCCUGUCUACACUUGCUGUGCAGA
ACAUCCGCUCACCUGUACAGCAGGCACAGACAGGCAGUCACAUGACAACCCAGCC
U
>DSSx6 peptide (SEQ ID NO: 13)
DSSDSSDSSDSSDSSDSS
>HABP peptide (SEQ ID NO: 14)
CγEPRRγEVAγELγEPRRγEVAγEL

What is claimed is:

1. A nucleic acid comprising a transgene encoding one or more miR-34-5p microRNAs (miRNAs) flanked by adeno-associated virus (AAV) inverted terminal repeats (ITRs).

2. The nucleic acid of claim 1, wherein the transgene encodes 2, 3, 4, or 5 miR-34-5p miRNAs, each miRNA having the sequence set forth in SEQ ID NO: 1.

3. The nucleic acid of claim 1 or 2, wherein each of the one or more miRNAs is encoded by the nucleic acid sequence set forth in SEQ ID NO: 2.

4. The nucleic acid of any one of claims 1 to 3, wherein the transgene further comprises a promoter.

5. The nucleic acid of claim 4, wherein the promoter is an RNA polymerase II (RNA pol II) or RNA polymerase III (RNA pol III) promoter.

6. The nucleic acid of claim 4 or 5, wherein the promoter comprises a chicken beta-actin (CBA) promoter or a U6 promoter.

7. The nucleic acid of any one of claims 1 to 6, wherein the AAV ITRs are AAV2 ITRs.

8. The nucleic acid of any one of claims 1 to 7 having the sequence set forth in SEQ ID NO: 4.

9. A recombinant adeno-associated virus (rAAV) comprising:
(i) the nucleic acid of any one of claims 1 to 8; and
(ii) an AAV capsid protein.

10. The rAAV of claim 9, wherein the AAV capsid protein is an AAV9 capsid protein.

11. A nucleic acid comprising a transgene encoding a miRNA inhibitor that inhibits expression of miR-214-3p in a subject flanked by adeno-associated virus (AAV) inverted terminal repeats (ITRs).

12. The nucleic acid of claim 11, wherein the miRNA inhibitor is a tough decoy (TuD) miRNA inhibitor.

13. The nucleic acid of claim 11 or 12, wherein the TuD miRNA inhibitor comprises 2 or 3 miR-214-3p binding sites.

14. The nucleic acid of claim 13, wherein each of the miR-214-3p binding sites comprises the sequence set forth in SEQ ID NO: 5.

15. The nucleic acid of any one of claims 11 to 14, wherein the transgene further comprises a promoter.

16. The nucleic acid of claim 15, wherein the promoter is an RNA polymerase II (RNA pol II) or RNA polymerase III (RNA pol III) promoter.

17. The nucleic acid of claim 15 or 16, wherein the promoter comprises a chicken beta-actin (CBA) promoter or a U6 promoter.

18. The nucleic acid of any one of claims 11 to 17, wherein the AAV ITRs are AAV2 ITRs.

19. The nucleic acid of any one of claims 11 to 18 having the sequence set forth in SEQ ID NO: 6.

20. A recombinant adeno-associated virus (rAAV) comprising:
(i) the nucleic acid of any one of claims 11 to 19; and
(ii) an AAV capsid protein.

21. The rAAV of claim 20, wherein the AAV capsid protein is an AAV9 capsid protein.

22. A method of inhibiting bone loss in a subject, the method comprising administering to a subject having bone loss a nucleic acid or rAAV as described in any of the preceding claims.

23. A method of increasing bone growth in a subject, the method comprising administering to a subject having increased bone density a nucleic acid or rAAV as described in any of the preceding claims.

24. A method for treating or preventing osteoporosis in a subject, the method comprising administering to a subject having, suspected of having, or at risk of having osteoporosis a nucleic acid or rAAV as described in any of the preceding claims.

25. The method of claim 22 or 23, wherein the administration comprises systemic administration or local administration, optionally wherein the administration comprises injection.

26. The method of any one of claims 22 to 24, wherein the subject is a mammal, optionally wherein the subject is a human.


SEQUENCE LISTING (34 sequences)

SEQ ID NO: 1 - RNA, 22 nt, Synthetic construct
  source 1..22: mol_type = other RNA
tggcagtgtcttagctggttgt

SEQ ID NO: 2 - RNA, 110 nt, Synthetic construct
  source 1..110: mol_type = other RNA
ggccagctgtgagtgtttctttggcagtgtcttagctggttgttgtgagcaatagtaaggaagcaatcagcaagtatactgccctagaagtgctgcacgttgtggggccc

SEQ ID NO: 3 - DNA, 329 bp, Synthetic construct
  source 1..329: mol_type = other DNA
tggcttccaagtgctggaggagtgtgtcatacctcggtagggtccactacacatctttctcccgcagcctctccatcttcctgtgactgcgggcgcctcagcctgggctggccagctgtgagtaattctttggcagtgtcttagctggttgttgtgagtattagctaaggaagcaatcagcaagtatactgccctagaagtgctgcacattgttgggccgagaaggaaaaggtcagaggtcagcaacgcccacacccctgagaggcgctggacttgcggagctgctcgaccatactggtgggtatgggatgctggggaggctggtgtta

SEQ ID NO: 4 - DNA, 2443 bp, Synthetic construct
  source 1..2443: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccagatcttggcttccaagtgctggaggagtgtgtcatacctcggtagggtccactacacatctttctcccgcagcctctccatcttcctgtgactgcgggcgcctcagcctgggctggccagctgtgagtaattctttggcagtgtcttagctggttgttgtgagtattagctaaggaagcaatcagcaagtatactgccctagaagtgctgcacattgttgggccgagaaggaaaaggtcagaggtcagcaacgcccacacccctgagaggcgctggacttgcggagctgctcgaccatactggtgggtatgggatgctggggaggctggtgttactgcaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 5 - DNA, 89 bp, Synthetic construct
  source 1..89: mol_type = other DNA
catcaacactgcctgtctggtcgtgcctgctgtcaagtattctggtcacagaatacaacactgcctgtctggtcgtgcctgctgtcaag

SEQ ID NO: 6 - DNA, 2378 bp, Synthetic construct
  source 1..2378: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgggatccgacggcgctaggatcatcaacactgcctgtctggtcgtgcctgctgtcaagtattctggtcacagaatacaacactgcctgtctggtcgtgcctgctgtcaagatgatcctagcgccgtcttttttaagcttgatccggtggtggtgcaaatcaaagaactgctcctcagtggatgttgcctttacttctaggcctgtacggaagtgttacttctgctctaaaagctgcggaattgtacccgcggccgatccaccggtcgccaccatctagcatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactagctcgacgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 2378 bp, Synthetic construct
  source 1..2378: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgggatccgacggcgctaggatcatcaacacaaccagctaagtcggacactgccacaagtattctggtcacagaatacaacacaaccagctaagtcggacactgccacaagatgatcctagcgccgtcttttttaagcttgatccggtggtggtgcaaatcaaagaactgctcctcagtggatgttgcctttacttctaggcctgtacggaagtgttacttctgctctaaaagctgcggaattgtacccgcggccgatccaccggtcgccaccatctagcatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactagctcgacgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 2509 bp, Synthetic construct
  source 1..2509: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccagatctttagttccataatgttttaatgtttaattctattgtgtgtttctctcctttccctttatccccctgtccttcccccctaaatcaccaaatctggaaaacaggctgattgtatctgtccctgagcaaaggaaacctgaaggacccaagggcctggctggacagagttgtcatgtgtctgcctgtctacacttgctgtgcagaacatccgctcacctgtacagcaggcacagacaggcagtcacatgacaacccagcctgaatgaccaccagccattgaaagaaagctgccctcacaacatagcatctacaccaagagctacaaccacagtgagggggttggggggcctggggtttgaaactgttggcttattaaaagaaaaactcgtatgtaatccctgcaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - RNA, 22 nt, Synthetic construct
  source 1..22: mol_type = other RNA
tggcagtgtcttagctggttgt

SEQ ID NO: 10 - RNA, 110 nt, Synthetic construct; identical to SEQ ID NO: 2
  source 1..110: mol_type = other RNA

SEQ ID NO: 11 - RNA, 22 nt, Synthetic construct
  source 1..22: mol_type = other RNA
acagcaggcacagacaggcagt

SEQ ID NO: 12 - RNA, 110 nt, Synthetic construct
  source 1..110: mol_type = other RNA
ggcctggctggacagagttgtcatgtgtctgcctgtctacacttgctgtgcagaacatccgctcacctgtacagcaggcacagacaggcagtcacatgacaacccagcct

SEQ ID NO: 13 - protein, 18 aa, Synthetic construct
DSSDSSDSSDSSDSSDSS

SEQ ID NO: 14 - protein, 19 aa, Synthetic construct
CEPRREVAELEPRREVAEL

SEQ ID NO: 15 - DNA, 89 bp, Synthetic construct
  source 1..89: mol_type = other DNA
catcaacacaaccagctaagtcggacactgccacaagtattctggtcacagaatacaacacaaccagctaagtcggacactgccacaag

SEQ ID NO: 16 - DNA, 89 bp, Synthetic construct
  source 1..89: mol_type = other DNA
tcatcttgtggcagtgtccgacttagctggttgtgttgtattctgtgaccagaatacttgtggcagtgtccgacttagctggttgtgtt

SEQ ID NO: 17 - DNA, 89 bp, Synthetic construct; identical to SEQ ID NO: 5
  source 1..89: mol_type = other DNA

SEQ ID NO: 18 - DNA, 89 bp, Synthetic construct
  source 1..89: mol_type = other DNA
tcatcttgacagcaggcacgaccagacaggcagtgttgtattctgtgaccagaatacttgacagcaggcacgaccagacaggcagtgtt

SEQ ID NO: 19 - DNA, 73 bp, Synthetic construct
  source 1..73: mol_type = other DNA
tcgagacaaccagctaagacactgccaacaaccagctaagacactgccaacaaccagctaagacactgccagc

SEQ ID NO: 20 - DNA, 73 bp, Synthetic construct
  source 1..73: mol_type = other DNA
ggccgctggcagtgtcttagctggttgttggcagtgtcttagctggttgttggcagtgtcttagctggttgtc

SEQ ID NO: 21 - DNA, 73 bp, Synthetic construct
  source 1..73: mol_type = other DNA
tcgagactgcctgtctgtgcctgctgtactgcctgtctgtgcctgctgtactgcctgtctgtgcctgctgtgc

SEQ ID NO: 22 - DNA, 73 bp, Synthetic construct
  source 1..73: mol_type = other DNA
ggccgcacagcaggcacagacaggcagtacagcaggcacagacaggcagtacagcaggcacagacaggcagtc

SEQ ID NO: 23 - DNA, 378 bp, Synthetic construct
  source 1..378: mol_type = other DNA
gtcttttatttcaggtcccagatcttggcttccaagtgctggaggagtgtgtcatacctcggtagggtccactacacatctttctcccgcagcctctccatcttcctgtgactgcgggcgcctcagcctgggctggccagctgtgagtaattctttggcagtgtcttagctggttgttgtgagtattagctaaggaagcaatcagcaagtatactgccctagaagtgctgcacattgttgggccgagaaggaaaaggtcagaggtcagcaacgcccacacccctgagaggcgctggacttgcggagctgctcgaccatactggtgggtatgggatgctggggaggctggtgttactgcaggggatccggtggtggtgc

SEQ ID NO: 24 - DNA, 444 bp, Synthetic construct
  source 1..444: mol_type = other DNA
gtcttttatttcaggtcccagatctttagttccataatgttttaatgtttaattctattgtgtgtttctctcctttccctttatccccctgtccttcccccctaaatcaccaaatctggaaaacaggctgattgtatctgtccctgagcaaaggaaacctgaaggacccaagggcctggctggacagagttgtcatgtgtctgcctgtctacacttgctgtgcagaacatccgctcacctgtacagcaggcacagacaggcagtcacatgacaacccagcctgaatgaccaccagccattgaaagaaagctgccctcacaacatagcatctacaccaagagctacaaccacagtgagggggttggggggcctggggtttgaaactgttggcttattaaaagaaaaactcgtatgtaatccctgcaggggatccggtggtggtgc

SEQ ID NO: 25 - RNA, 102 nt, Synthetic construct
  source 1..102: mol_type = other RNA
ccagctgtgagtaattctttggcagtgtcttagctggttgttgtgagtattagctaaggaagcaatcagcaagtatactgccctagaagtgctgcacattgt

SEQ ID NO: 26 - RNA, 23 nt, Synthetic construct
  source 1..23: mol_type = other RNA
tcttgtgttgctgtgtttgctgt

SEQ ID NO: 27 - RNA, 23 nt, Synthetic construct
  source 1..23: mol_type = other RNA
gatcctactaattatcctgctgt

SEQ ID NO: 28 - RNA, 23 nt, Synthetic construct
  source 1..23: mol_type = other RNA
tattttccacagaaacactgcct

SEQ ID NO: 29 - RNA, 23 nt, Synthetic construct
  source 1..23: mol_type = other RNA
tatccaagtacggatcactgcca

SEQ ID NO: 30 - protein, 14 aa, Synthetic construct
  VARIANT 9..14: note = May be absent
DDDDDDDDDDDDDD

SEQ ID NO: 31 - protein, 5 aa, Synthetic construct
RGBYK

SEQ ID NO: 32 - protein, 8 aa, Synthetic construct
DDDDDDDD

SEQ ID NO: 33 - protein, 5 aa, Synthetic construct
GGGGS

SEQ ID NO: 34 - protein, 4 aa, Synthetic construct
  VARIANT 1..4: note = The entire sequence of amino acids 1-4 can be repeated one or more times
GGSG